(19) United States
(12) Patent Application Publication
(10) Pub. No.: US 2022/0249801 A1
(43) Pub. Date: Aug. 11, 2022

(54) BREATHING MEDITATION INDUCTION DEVICE COMBINED WITH HEADPHONES FOR SENSING BRAIN WAVE SIGNALS, BREATHING MEDITATION INDUCTION SYSTEM FOR DISPLAYING AND STORING BRAIN WAVE SIGNALS BY USING SAME, AND SYSTEM FOR BRAIN WA VE SIGNAL MANAGEMENT THROUGH MIDDLE MANAGER

(71) Applicant: HEALLIAN CO., LTD., Gyeonggi-do (KR)

(72) Inventor: In-Tea LEE, Gyeonggi-do (KR)

(21) Appl. No.: 17/608,344

(22) PCT Filed: May 4, 2020
(86) PCT No.: PCT/KR2020/005909

§ 371 Date: Jan. 4, 2022

(30) Foreign Application Priority Data
May 2, 2019 (KR) 10-2019-0051803

(51) Int. Cl. A61M 21/02

(52) U.S. Cl. CPC A61M 21/02; A61B 17/24

(57) ABSTRACT

A breathing meditation inducing device combined with headphones capable of measuring EEG, including a main body mounted on the user's face, and a control box electrically coupled to the main body and providing a driving signal to the main body is provided where the main body includes first and second opening and closing means for opening and closing the nostril of the user's nose, first and second driving means for driving the first and second opening and closing means, respectively, and mounting means for mounting the main body on the face In the headphone and breathing meditation inducing device capable of EEG measurement, characterized in that the control box is further provided with a memory card holder for inserting a memory card, the headphone installation part has an inner surface.

### BACKGROUND

The present invention relates to a breathing meditation inducing device combined with headphones for sensing brain wave signals (or EEG signals) which enables the user's psychotherapy to be effective and displays EEG signals, and a breathing meditation induction system for displaying and storing EEG signals using the device. As a result, by downloading an application for a breathing meditation induction system through a personal terminal capable of wired and wireless communication and a combined headphone breathing meditation induction device, mental stability and psychological treatment can be achieved using the headphone combined breathing meditation induction device connected through Bluetooth communication. It relates to a breathing meditation induction device combined with headphones capable of displaying the enabled EEG, a breathing meditation induction system that displays and stores EEG signals using the device, and an EEG signal management system through an intermediate manager.

### BACKGROUND ART

From ancient times, in the East including Korea, China, and India, it has been recognized that a person's breathing method has a great influence on the health of the person's mind and body. Therefore, the breathing method occupies a large proportion in the mind and body training methods in various religions and martial arts fields.

Currently, abdominal breathing, chest breathing, single breathing, and alternating breathing are widely known as breathing methods. Here, the term “alternative breathing” is a type of breathing method generally performed by yogis, and refers to a method of performing breathing by alternately selecting any one of two nostrils. Alternating respiration, because breathing is done through one nostril, the flow of air during inhalation and exhalation is accelerated, thereby clearing the respiratory tract from the nose to the bronchial tubes and lungs, and is effective in neurological diseases such as insomnia, hypertension, depression and anxiety It is known that the sympathetic and parasympathetic nerves, as well as all the functions of the mind and body, maintain a balance and have a good effect on chronic diseases caused by constitutional imbalance. Therefore, alternating breathing is widely used as a breathing method for mind-body practitioners, either alone or in parallel with abdominal breathing or single breathing.

In general, when the mind-body practitioner performs alternating breathing, a method of selectively opening and closing the nasal vestibule, which is the front part of the nasal cavity, is adopted using a finger. However, this method of using the fingers of the human body is based on the individual conscious activity of the meditator, so it is of course an obstacle to concentrating on mind and body performance. There is a risk of performing incorrect alternating breathing. It has been reported that the wrong breathing method can cause headaches or impair mental and physical stability.

In addition, in other breathing methods including abdominal breathing, it is very difficult for the user to properly control the inhalation and exhalation, and as a result, as the user breathes according to the wrong breathing method, there are many cases where health is rather harmed.

Prior art literature: Republic of Korea Patent Registration No. 10-1463993 (registered on Nov. 14, 2014) (Title of the invention: Respiratory control device)

### SUMMARY OF THE INVENTION

Accordingly, the present invention has been created in view of the above circumstances, and it provides a device for inducing a breathing meditation inducing both headphones and a headphone that senses an EEG signal that allows an effect on a user's psychotherapy and displays an EEG, and the device. It relates to a breathing meditation induction system that displays and stores brainwave signals using headphones, which are connected through Bluetooth communication by downloading an application for the breathing meditation induction system through a headphone-combined breathing meditation induction device and a personal terminal capable of wired and wireless communication. It is to provide a breathing meditation induction device combined with headphones that can display brain waves that enable mental stability and psychological treatment using the combined breathing meditation induction device, and a breathing meditation induction system that displays and stores brain wave signals using the device.

Another object of the present invention is to provide an environment in which data such as heart rate, oxygen saturation, etc. can be measured as well as EEG signals to an individual, and display EEG measurement signals to provide an environment in which the individual can check the physical condition accordingly.

To provide an EEG measurement signal management system to store, according to an embodiment of the present invention for realizing the above object, a breathing mediation induction device combined with headphones for sensing brain wave signals comprises:

a body mounted on the user's face,
a control device being electrically coupled to the body, and that provides a driving signal to the body,
wherein the body includes first and second opening and closing means for opening and closing the nostril of the user's nose;
first and second driving means for driving the first and second opening and closing means, respectively; and a mounting means for mounting the body on a specific part of the face,
wherein headphones are integrally coupled to the main body and include an earmuff for covering the user's ear and having a headphone installation unit for covering the user's head,
wherein a pair of EEG measuring electrodes capable of measuring EEG, which is a kind of biosignal, is installed on the inner surface of the headphone installation unit,
wherein one side of the headphone includes a sensing means for measuring EEG composed of one electrode for grounding, and includes a USB interface for coupling to a personal computer or smart phone,
wherein the A breathing mediation induction device senses an EEG signal, and is connected to a personal computer or smart phone, and transmits the sensed EEG signal to the personal computer or smart phone by operating the user's EEG according to the measurement request signal using the personal computer or smart phone.

According to the present invention having the above configuration, the user wears the breathing meditation inducing device combined with headphones according to the present invention on the face, and detects EEG through the sensing device for EEG sensing that is in contact with the forehead inside the headphone device over both eyes, By executing the breathing meditation of the breathing meditation induction device combined with headphones, it is possible to see the changes in the brain waves before and after using the device in a three-dimensional waveform and in a bar graph in various ways.

In addition, by downloading the application for the breathing meditation induction system through a personal terminal capable of wired or wireless communication, and using a breathing meditation induction device for headphones connected through Bluetooth communication, the brain waves are detected, and the changes in the brain waves are drawn into a three-dimensional waveform and a bar graph. It has the effect of enabling mental stability and psychological treatment by being able to blow in a variety of ways.

In addition, the individual can promote mental and physical stability by clicking on the data obtained through the analysis result to execute the appropriate content for the data, and after executing the content (provided by the operator of the present invention), various signals such as EEG signals Since it can be measured and analyzed, there is an effect that the improvement effect of mental and physical conditions such as mental and physical stability through the contents of the present invention can be visually confirmed.

In addition, according to the present invention, since the middle manager who manages a plurality of individuals can manage specific individuals by field, meaningful data can be collected for each group according to the ability of the middle manager, and also the manager of the present invention can manage big data It has the effect of being able to serve as a manager who can collect and provide a platform to middle managers.

In addition, according to the present invention, an individual can additionally collect bio-signal-related data such as heart rate, blood vessel age, oxygen saturation, and electrocardiogram as well as brain waves, and through this, check the current state of the body through bio-signal measurement There is also the effect of being able to check the state of one's body management by uploading it.

### BRIEF DESCRIPTION OF DRAWINGS

FIG. 1 and FIG. 2 are perspective views schematically showing a breathing meditation inducing device combined with headphones according to the present invention.

FIG. 3 is a block diagram of the respiratory noun induction device of FIG.

FIG. 4 is a diagram illustrating an example of an interface screen displayed on a personal terminal in a breathing meditation induction system using a breathing meditation induction device combined with headphones that can display brain waves by being connected to a personal terminal and Bluetooth communication according to the present invention.

FIG. 5 is a diagram illustrating an example of a pop-up window displayed when my EEG data file is touched in FIG. 4.

FIG. 6 is a view showing an example of a screen and a 3D (three-dimensional) graph screen after clicking a frequency band in the pop-up window displayed in FIG. 5.

FIG. 7 is a graph of changes in brain index displayed when a brain function is selected in the pop-up window displayed in FIG. 5.

FIG. 8 is an interface screen illustrating an example of an EEG preparation screen displayed when an EEG measurement is selected on the screen of FIG. 4.

FIG. 9 is an example of a screen where EEG measurement starts when the user contacts the EEG sensor of the breathing meditation inducing device combined with headphones in FIG. 8 and touches the reference signal frequency measuring unit (ground) to the earlobe is a diagram showing.

FIG. 10 is a view similar to FIG. 9 as showing an interface screen in which EEG measurement is continuing in FIG. 8.

FIG. 11 is a view showing an example of the EEG measurement completion screen in which the EEG measurement in FIG. 8 is completed.

FIG. 12 is a view showing an example of the EEG measurement result screen in which the EEG measurement in FIG. 8 is completed.

FIG. 13 is a diagram schematically illustrating a process of storing data in a server and a personal terminal when the data storage button is selected in FIG. 12.

FIG. 14 is a detailed explanatory diagram of a side menu in the EEG measurement result screen of FIG. 12.

FIG. 15 is a diagram illustrating an example of a comparison result data screen in the EEG measurement result screen of FIG. 12.

FIG. 16 is an interface screen for setting brain index measurement items displayed on a personal terminal in a breathing meditation induction system using a breathing meditation induction device combined with headphones that can display brain waves by being connected through Bluetooth communication with a personal terminal according to the present invention. It is a drawing showing an example.

FIG. 17 is an example of a more specific brain index display interface screen displayed on a personal terminal in a breathing meditation induction system using a breathing meditation induction device combined with headphones that can display brain waves by being connected through Bluetooth communication with a personal terminal according to the present invention. the drawing shown.

FIG. 18 is a conceptual diagram explaining the process for saving EEG measurement results to a cloud database: the results measured by a personal measurement device in a breathing meditation induction system using a breathing meditation induction device combined with headphones that can display brain waves by being connected through Bluetooth communication with a personal terminal according to the present invention.

FIG. 19 is a conceptual diagram schematically illustrating a process of accessing a database of a cloud server through a personal terminal connected to the cloud server through a communication network in FIG. 18.

FIG. 20 is a brain index graph and three-dimensional brain wave data before and after use provided in a breathing meditation induction system using a breathing meditation induction device combined with headphones that can display brain waves by being connected through Bluetooth communication with a personal terminal according to the present invention; It is a conceptual diagram schematically showing the process of analyzing the brain index state and outputting it as a reporter.

FIG. 21 is a personal terminal and a personal terminal according to the present invention connected through Bluetooth communication in the breathing meditation inducing system using a headphone and breathing meditation inducing device capable of displaying brain waves in the state of accessing the cloud server through a personal terminal, database for each member It is a conceptual diagram showing the state to be managed and displayed.

FIG. 22 is a breathing meditation inducing device that can measure actual brain waves in a breathing meditation inducing system using a headphone and breathing meditation inducing device that can display brain waves by being connected through Bluetooth communication with a personal terminal according to the present invention. It is a figure which shows an example of a state.

FIG. 23 is a diagram showing an example of a management platform system of EEG measurement signal big data through an individual and an intermediate manager in a breathing meditation induction system using a breathing meditation induction device combined with headphones that can display brain waves by being connected through Bluetooth communication with a personal terminal according to the present invention.

FIG. 24 is a diagram illustrating an example of a screen for performing an EEG signal and a heart rate measurement result in the personal terminal of FIG. 23.

FIG. 25 is a diagram illustrating an example of a screen that displays an index such as a stress index while performing an EEG signal and a heart rate measurement result in the personal terminal of FIG. 23.

FIG. 26 is a flowchart illustrating an example of a process of measuring an EEG signal and a heart rate in an individual and/or middle manager terminal in FIG. 23.

FIG. 27 is a flowchart illustrating an example of a login process for performing EEG signal and heart rate measurement in the middle manager terminal in FIG. 23.

FIG. 28 is a flowchart illustrating an example of a billing process after measuring an EEG signal and a heart rate in the middle manager terminal in FIG. 23.

### DETAILED DESCRIPTION OF THE INVENTION

In order to accomplish the above objects, according to an aspect of the present invention, there is provided a breathing mediation induction device combined with headphones for sensing brain wave signals comprising:

a body mounted on the user's face,
a control device being electrically coupled to the body, and that provides a driving signal to the body,
wherein the body includes first and second opening and closing means for opening and closing the nostril of the user's nose;
first and second driving means for driving the first and second opening and closing means, respectively; and a mounting means for mounting the body on a specific part of the face,
wherein headphones are integrally coupled to the main body and include an earmuff for covering the user's ear and having a headphone installation unit for covering the user's head,
wherein a pair of EEG measuring electrodes capable of measuring EEG, which is a kind of biosignal, is installed on the inner surface of the headphone installation unit,
wherein one side of the headphone includes a sensing means for measuring EEG composed of one electrode for grounding, and includes a USB interface for coupling to a personal computer or smart phone,
wherein the A breathing mediation induction device senses an EEG signal, and is connected to a personal computer or smart phone, and transmits the sensed EEG signal to the personal computer or smart phone by operating the user's EEG according to the measurement request signal using the personal computer or smart phone.

Preferably, wherein the sensing means for measuring EEG is located so as to be in contact with the upper forehead of both eyes and made of electrically conductive materials is a device for inducing breathing meditation combined with headphones for sensing EEG signals.

In another aspect, there is provided a breathing mediation induction system for displaying and storing EEG signal comprising:

a body mounted on the user's face,
a control device being electrically coupled to the body, and that provides a driving signal to the body,
wherein the body includes first and second opening and closing means for opening and closing the nostril of the user's nose;
first and second driving means for driving the first and second opening and closing means, respectively; and a mounting means for mounting the body on a specific part of the face,
wherein headphones are integrally coupled to the main body and include an earmuff for covering the user's ear and having a headphone installation unit for covering the user's head, wherein in an application through a personal terminal, when the user terminal selects the EEG measurement function through touch input, the EEG through the sensing means for EEG measurement of the breathing meditation inducing device combined with headphones is measured and then the results is transmitted to the personal terminal or a manager's computer (server),
wherein the EEG measurement and display program is loaded as an application on the personal terminal, and according to the user's selection, it measures EEG or displays the measured result data, and at the same time provides content according to the user's requirements,
wherein when the application is used, the user terminal controls EEG measurement preparation processes so as to be performed the EEG measurement and display functions being provided for a predetermined time, and controls the EEG measurement result data to display the EEG measurement result data in a three-dimensional graph or bar graph form, and when the EEG measurement result is displayed, the comparison data of the past data and the present data is controlled to be displayed on one screen according to the user's request.

Preferably, wherein a user terminal is connected to a personal terminal with a built-in breathing meditation induction device for brain wave measurement and a program that can display and store EEG signals via Bluetooth, and the personal terminal is connected to a computer server for storing EEG signals, and

wherein by operating the app of the personal terminal, the EEG signal is measured, the degree of change is displayed, in the measurement of one's own state through the brain index measurement graph, after a predetermined time after using the psychotherapy device for the brain index measurement bar graph, and the display result can be stored in the computer (server).

More preferably, wherein a user terminal is connected to a personal terminal with a built-in psychotherapy device capable of EEG measurement and a program capable of displaying and storing EEG signals through Bluetooth, and the personal terminal is connected to the EEG signal storage computer server, and

wherein by operating the application of the personal terminal, measuring EEG signals, and grasping one's own state through the brain index measurement bar graph, the brain index measurement bar graph displays the user's mental state into various items by type and when selecting the corresponding item by touch or voice, one or more contents are downloaded thorough a computer (server), and then the specific content can be played by selecting it by touch or voice.

In another aspect, there is provided a personal terminal for displaying and storing EEG signals in which it includes a Bluetooth, and an application for breathing meditation induction, the application can be downloads through a wireless communication network, and is connected through a wireless communication network and can controls a headphone through a voice or touch input method of the application,

wherein based on the data inputted and transmitted according to the input requirements of the device application, a self-diagnosis process is performed, the result is transmitted as a score through a personal terminal, and content for breathing meditation induction or psychotherapy according to the self-diagnosis score result is provided,

wherein the content is extracted from the database and supplied to the headphone and breathing meditation induction device connected to the personal terminal in a streaming manner,

wherein, in the application through the personal terminal, when the user selects the EEG function through a touch input, the EEG is measured through the EEG sensor of the psychotherapy device and the result is stored in the storage unit of the personal terminal,

wherein the EEG measurement and display program is loaded as an application on the personal terminal, and according to the user's selection, it measures EEG or displays the measured result data, and at the same time provides content according to the user's requirements,

wherein when running the application, and the user executes the EEG measurement preparation process to perform the EEG measurement and display function provided by the app, then controls the EEG measurement to be performed for a predetermined time, and displays the EEG measurement result data as a three-dimensional graph or EEG signal display, when the EEG measurement result is displayed, the comparison data of the past data and the present data is controlled to be displayed on one screen according to the user's request.

Preferably, wherein, in the state that the user is connected via Bluetooth to a personal terminal with a built-in breathing meditation induction device and a program that can display and store EEG signals,

wherein the user terminal starts the application of the personal terminal, measures the EEG signal, and determines one's own state through the brain index measurement bar graph, but after using the headset and breathing meditation inducing device for a specific item as a result of the brain index measurement, the brain index after a predetermined time, wherein it is possible to display the degree of change through the measurement graph.

More preferably, wherein the items displayed on the brain index measurement bar graph are frequency data measured by the EEG sensing means, and include delta waves, theta waves, alpha waves, SMR waves, M_beta waves, H_beta waves, beta waves, and gamma waves, correspondingly, the brain index bar graph is divided into right brain frequency band and left brain frequency band, respectively, and converted and displayed.

More preferably, wherein the items displayed on the brain index measurement bar graph are frequency data measured by the EEG sensing means, and include delta waves, theta waves, alpha waves, SMR waves, M_beta waves, H_beta waves, beta waves, and gamma waves, correspondingly, the brain index bar graph is divided into right brain frequency band and left brain frequency band, respectively, and converted and displayed, and the index graph is displayed as an additional item.

In another aspect, there is provided a breathing meditation induction system to display and store the EEG signal in which it includes a Bluetooth, and an application for breathing meditation induction, the application can be downloads through a wireless communication network, and is connected through a wireless communication network and can controls a headphone through a voice or touch input method of the application,

wherein based on the data inputted and transmitted according to the input requirements of the device application, a self-diagnosis process is performed, the result is transmitted as a score through a personal terminal, and content for breathing meditation induction or psychotherapy according to the self-diagnosis score result is provided,

wherein the content is extracted from the database and supplied to the headphone and breathing meditation induction device connected to the personal terminal in a streaming manner,

wherein, in the application through the personal terminal, when the user selects the EEG function through a touch input, the EEG is measured through the EEG sensor of the psychotherapy device and the result is stored in the storage unit of the personal terminal,

wherein the EEG measurement and display program is loaded as an application on the personal terminal, and according to the user's selection, it measures EEG or displays the measured result data, and at the same time provides content according to the user's requirements,

wherein when running the application, and the user executes the EEG measurement preparation process to perform the EEG measurement and display function provided by the app, then controls the EEG measurement to be performed for a predetermined time, and displays the EEG measurement result data as a three-dimensional graph or EEG signal display, when the EEG measurement result is displayed, the comparison data of the past data and the present data is controlled to be displayed on one screen according to the user's request.

In another aspect, there is provided an EEG measurement signal management system through the middle manager server in which it includes a Bluetooth, and an application for breathing meditation induction, the application can be downloads through a wireless communication network, and is connected through a wireless communication network and can controls a headphone through a voice or touch input method of the application,

wherein based on the data inputted and transmitted according to the input requirements of the device application, a self-diagnosis process is performed, the result is transmitted as a score through a personal terminal, and content for breathing meditation induction or psychotherapy according to the self-diagnosis score result is provided,

wherein the content is extracted from the database and supplied to the headphone and breathing meditation induction device connected to the personal terminal in a streaming manner,

wherein it includes other middle manager servers divided by multiple job types, and the middle manager server stores information about a number of individuals and manages this information. Middle manager server that executes star statistics processing function,

wherein it includes an EEG measurement signal processing system manager server or cloud server in which the signal measured through the headphone-combined breathing meditation inducing device is connected through a network communication network through a personal terminal and uploaded, and the uploaded data is divided into a database and separated into a separate database, wherein the EEG measurement signal manager server distinguishes and displays the EEG measurement signal and the related graphic screen related to heart rate processing through the interface screen,

wherein the middle manager server is a computer server for schools, government offices, private institutes, and insurance companies, and plays a role as a manager.

Preferably, wherein the middle manager server statistically classifies information on heart rate, pulse rate, oxygen saturation, glycemic index, and blood vessel age as well as related statistical screens related to EEG signal and heart rate processing through an interface screen. Or an EEG measurement signal management service system through an intermediate manager server that processes EEG measurement signals using a breathing meditation inducing device combined with headphones, characterized in that it is displayed in 2D graphics.

In another aspect, there is provided an EEG measurement signal management method through the middle manager server in which it includes a Bluetooth, and an application for breathing meditation induction, the application can be downloads through a wireless communication network, and is connected through a wireless communication network and can controls a headphone through a voice or touch input method of the application,

wherein based on the data inputted and transmitted according to the input requirements of the device application, a self-diagnosis process is performed, the result is transmitted as a score through a personal terminal, and content for breathing meditation induction or psychotherapy according to the self-diagnosis score result is provided,

wherein the content is extracted from the database and supplied to the headphone and breathing meditation induction device connected to the personal terminal in a streaming manner,

wherein it comprises:

EEG, heart rate measurement preparation step,
wherein in the case of being ready, the multi-step EEG/heart rate measurement is performed through the breathing meditation inducing device combined with headphones, afterwards, the EEG and heart rate measurement result data is saved, uploaded to the EEG measurement signal processing system manager server, and the analysis data is sent to the individual so as to provide for copying, printing, and sharing; and
wherein if the individual is an individual belonging to the middle manager, including the step of storing the result data in the middle manager member database, EEG measurement signal management method using an EEG measurement signal management system through an intermediate manager, and the measured EEG signal and heart rate data are displayed through the interface screen of the personal terminal or PC terminal or tablet terminal.

Preferably, wherein if the individual is an individual belonging to a specific middle manager, displaying an administrator screen when logging in with the middle manager ID and PW to which the individual belongs;

after that, the step of requesting member statistical data search;
after that, check the billing status for the middle manager to determine whether the billing is complete;
after that, if the billing is completed, the member statistical data requested by the middle manager is displayed, and then the middle manager manages the information about the individual he is managing by performing inquiry, search, and editing processes;
wherein if the billing is not completed, determining the payment completion status, and providing a service when the payment is completed through a bank server or a billing server.

More preferably, wherein the middle manager server statistically divides and displays information about heart rate, pulse rate, oxygen saturation, glycemic index, and blood vessel age as well as related statistical screens related to EEG signal and heart rate processing through the interface screen.

More preferably, wherein the electrocardiogram is performed through an electrocardiogram sensor mounted on the ear.

More preferably, wherein the measurement result index is displayed in 2D or 3D graphics, and when the corresponding graphic is clicked, content corresponding to the corresponding measurement result is displayed, and when the content is clicked, the corresponding content is immediately executed.

### MODE FOR INVENTION

Hereinafter, an embodiment according to the present invention will be described with reference to the drawings. However, the embodiments described below are illustratively shown as one preferred embodiment of the present invention, and the examples of these embodiments are not intended to limit the scope of the present invention. The present invention can be implemented with various modifications within the scope that does not depart from its technical spirit.

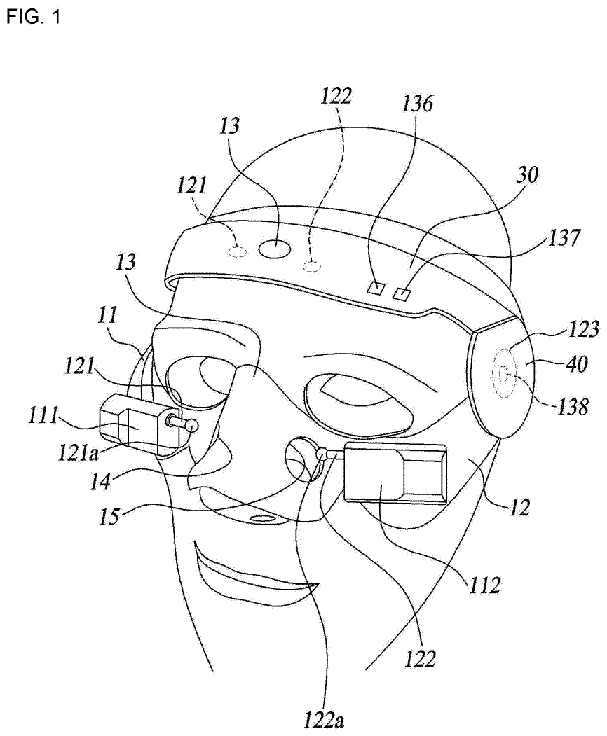

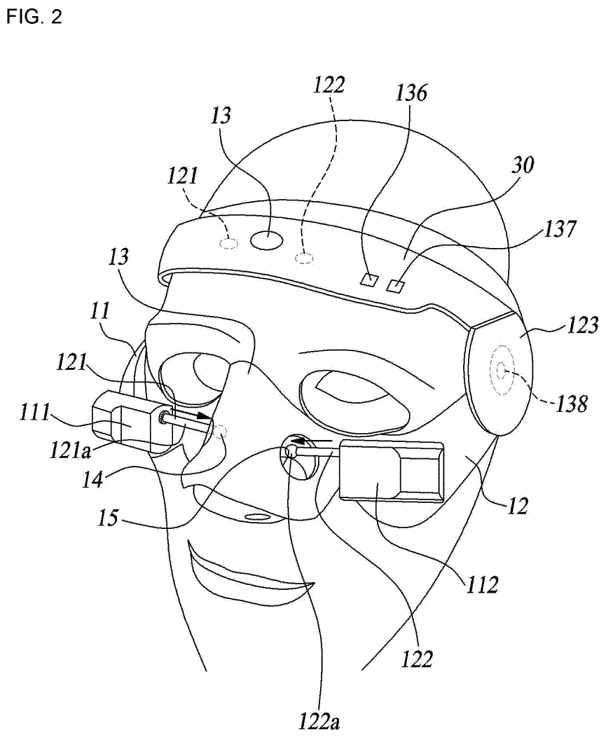

FIG. 1 and FIG. 2 are perspective views schematically showing a breathing meditation inducing device combined with headphones according to the present invention.

The headphone device 30 is integrally coupled to and connected to the headphone and breathing meditation inducing device via the ear cover unit 40. The body parts 11 and 12 of the breathing meditation inducing device are each having motors 111 and 112 fixedly installed, and at the ends of the motors 111 and 112, a movable bar (opening and closing member) 121 and 122 having contact rods 121a and 122a is provided. installed Unexplained reference numeral 13 denotes a nose cover part, and is connected to the body parts 11 and 12. The nasal cover portion 13 has perforations 14 and 15 formed in portions corresponding to the left and right nasal passages of the nose.

Reference numeral 13, not described, denotes a sensing unit (a part for sensing by touching a finger), reference numerals 121 and 122 denote EEG measuring electrodes, and 123 EEG measuring ground electrodes.

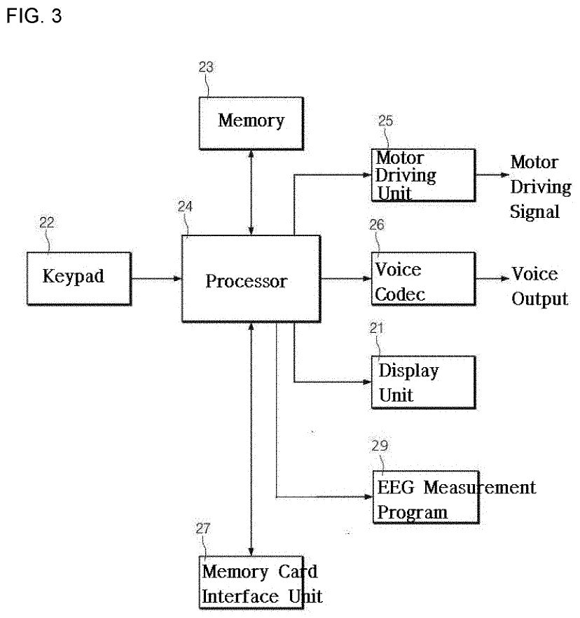

FIG. 3 is a block diagram schematically showing the circuit configuration of the personal terminal 200. The personal terminal 200 includes a keypad 22 on which various keys are installed, a memory 23 for storing various data for control operation, a processor 24 for controlling the entire device, and a processor in addition to the display unit 21 described above. Based on the control data from (24), the digital voice data applied from the motor driving unit 25 and the processor 24 for outputting a driving signal for driving the motors 111 and 112 of the main body of the breathing meditation induction device combined with headphones is an analog voice signal. It is configured with a voice codec 26 for converting and outputting, and a memory card interface unit 27 for interfacing with the memory card 230. These circuits are operated by battery power, not shown.

In the above configuration, the configuration for use of the memory card 230 may be selectively used as needed, and the memory 23 and the internal memory of the processor 24 may be used. In addition, a connector with an external device may be additionally provided for data download or the like. That is, the circuit portion of the personal terminal 200 is not limited to a specific one, and some components may be excluded or added as necessary.

The processor 24 sets the entire device to an operating state when the user selects the power button. Then, the user selects the operation mode by using the meditation music selection button (not shown), the breathing method selection button (not shown), the operation time and number of breaths per minute selection button (not shown) and the sub-menu selection button (not shown). When set, based on this, it outputs a motor drive signal through the motor driving unit 25 to drive and control the motor of the main body of the breathing meditation induction device combined with headphones. It is output through the earphone of the main body of the breathing meditation induction device.

In the device, as an operation mode selectable by the user, there are, for example, abdominal breathing, alternating breathing, negative breathing, and positive breathing.

1. Abdominal Breathing Mode

In the abdominal breathing mode, the processor 24 simultaneously drives the motors 111 and 12 provided in the main body of the breathing meditation induction device combined with headphones to first move the two opening and closing members (in the form of protruding rods) 121 and 122 to the left and right sides of the main body at the same time. It controls to move, and after a certain period of time, a repeating operation of simultaneously returning and moving them back toward the main body is executed. At this time, the time interval for reciprocating the opening and closing members 121 and 122 is executed based on the operation time and the number of respirations per minute set by the user through the button for selecting the number of breaths per minute and the sub-menu selection button.

In addition, the processor 24 drives the motor and provides the meditation music selected by the user to the earphone of the main body through the voice codec. Of course, in this case, if there is a guide voice to be guided to the user in relation to the breathing operation, it is provided with the earphone along with the meditation music.

The above operation is performed during the operation time set by the user through the operation time and the number of breaths per minute selection button and the sub-menu selection button.

2. Alternate Breathing Mode

In the alternating breathing mode, the processor 24 alternately drives the motors 111 and 112 provided in the body. For example, the processor 24 first moves one opening/closing member 121 and 122 to the left so as to protrude from the main body, and the other opening/closing member 121 and 122 moves to the right side of the main body, and again after a certain period of time, one opening/closing member (121, 122) is moved to the right side of the main body, and the other opening/closing member is repeatedly performed alternating motion of moving to the left side of the main body.

Even in this case, the time interval for alternately reciprocating the opening and closing members 121 and 122 is executed based on the operation time and the number of respirations per minute set by the user through the button for selecting the number of breaths per minute and the button for selecting the sub-menu.

In addition, the processor 24 is executed for the time and meditation music selected by the user along with the driving of the motors 111 and 112. That is, in this mode, the opening and closing members 121 and 122 alternately move from left to right or from right to left.

3. Negative Breathing Mode

In the negative breathing mode, the processor 24 fixes one opening/closing member 121 corresponding to the user's right nostril in a state moved to the left, and reciprocating the opening/closing member 122 corresponding to the user's left nostril at regular time intervals. move

To this end, the processor 24 first drives the motor 112 corresponding to the opening/closing member 122 corresponding to the right nostril to move the opening/closing member 122 to one side. Then, the processor 24 drives the motor 111 to move the corresponding left opening/closing member 121 to one side, and then repeats the reciprocating operation of moving the opening/closing member 121 to the other side after a predetermined time.

Even in this case, the time interval for reciprocating the opening/closing member is executed based on the operation time and the number of respirations per minute set by the user through the button for selecting the number of breaths per minute and the button for selecting the sub-menu.

In addition, the processor 24 provides the user-selected meditation music and guidance voice to the earphone of the main body through the voice codec 26 as well as driving the motor. The above operation is performed during the operation time set by the user through the operation time and the number of breaths per minute selection button and the sub-menu selection button.

4. Positive Breathing Mode

In the positive breathing mode, the processor 24 fixes the opening/closing member 121 corresponding to the user's left nostril in a state moved to the right, and reciprocating the opening/closing member 122 corresponding to the user's right nostril at regular time intervals.

To this end, the processor 24 first drives the motor corresponding to the opening and closing member corresponding to the left nostril to move the opening and closing member to the right. Then, the processor 24 drives the motor to move the opening/closing member corresponding to the right nostril to the left side of the body, and then repeats the reciprocating operation of moving the opening/closing member to the right side of the body after a predetermined time.

Even in this case, the time interval for reciprocating the opening/closing member is executed based on the operation time and the number of respirations per minute set by the user through the button for selecting the number of breaths per minute and the button for selecting the sub-menu.

In addition, the processor 24 provides the user-selected meditation music and guidance voice to the earphone of the main body through the voice codec along with driving the motor.

The above operation is performed during the operation time set by the user through the operation time and the number of breaths per minute selection button and the sub-menu selection button.

In the above operation, when the opening/closing member is moved to the right side of the body, the opening/closing opening of the opening/closing member closes the nostril of the nose and the corresponding nostril is blocked, thereby blocking exhalation and inhalation through the corresponding nostril, and the opening/closing member moves to the left side of the body When moved, the opening/closing port of the opening and closing member that shielded the nostril moves downward, and the nostril is opened again in a normal state, so that exhalation and inhalation can freely come and go through the nostril.

Therefore, when the above-described device is used, the practitioner who trains mind and body through the breathing method can efficiently perform various breathing methods without using his or her fingers, so that he can concentrate on the performance of the mind and body.

In addition, in the above-described device, since the processor drives the opening and closing member at a preset precise time interval, not only the performer with abundant performance experience but also the performer who is new to the breathing method can safely perform various breathing methods.

In addition, in the above-described device, meditation music and the like are provided to the meditator in addition to driving the opening and closing member, so that the performance of the meditator can be improved.

An embodiment according to the present invention has been described above. However, the present invention is not limited to the above-described embodiments and can be implemented with various modifications within the scope without departing from the technical spirit of the present invention.

In the above embodiment, it has been described that a motor is used as a driving means for driving the opening/closing member, but it is also possible to use an electromagnet or a hydraulic or pneumatic actuator as the driving means.

The breathing control device according to the present invention allows the user to conveniently and effectively perform various breathing methods just by wearing it on the face. In addition, since the respiratory control device according to the present invention opens and closes the user's nostrils at precise time intervals, even a user who has no experience in breathing methods can safely execute various breathing methods without additional training.

In addition, the present invention is not limited to the above-described embodiments, and various modifications may be made within the scope without departing from the technical spirit of the present invention.

For example, in the above embodiment, it has been described that the USB interface is used when combining the headphone and breathing meditation induction device with a personal computer or smart phone, but the coupling method is also preferably a wireless communication method such as Bluetooth. have.

In addition, the headphone-combined breathing meditation inducing device can be preferably applied to a method of directly controlling from an external device using a wired/wireless communication method.

Breathing meditation induction device combined with headphones according to the present invention comprises a body mounted on the user's face.

It is configured to include a control device that provides a driving signal to the body, and is electrically coupled to the body,

The body includes first and second opening and closing means for opening and closing the nostril of the user's nose; first and second driving means for driving the first and second opening and closing means, respectively; and a mounting means for mounting the body on a specific part of the face.

It is a combined headphone breathing meditation inducing device, characterized in that it is connected to the main body and includes an ear cover that covers the user's ear and a headphone having a headphone installation part that wraps around the user's head is integrally coupled.

The control device can be installed in the body.

It may be provided in the form of an application in a separate device, such as a smartphone or a personal portable terminal.

A pair of EEG measuring electrodes capable of measuring EEG, which is a kind of biosignal, is installed on the inner surface of the headphone installation unit.

The headphone part includes a sensing means for EEG measurement consisting of a pair of EEG measuring electrodes capable of measuring EEG, which is a kind of biosignal, and one grounding electrode, and it includes a USB interface for coupling to a personal computer or smart phone.

It is connected to a personal computer or smart phone, and operates according to the measurement request signal of the user using the personal computer or smart phone, characterized in that the sensed EEG signal is transmitted to the personal computer or smart phone.

The sensing means for measuring EEG is positioned so as to be in contact with the upper forehead of both eyes of the user, and is made of a material through which electricity can pass.

In an embodiment of the present invention, preparing for measuring EEG in a state in which a personal terminal and a breathing meditation inducing device combined with headphones are connected via Bluetooth communication (not limited thereto), and EEG of a sensing means for EEG measurement in contact with the user's forehead It is possible to start sensing EEG signals through the measuring electrode. Of course, this can be done while the grounding electrode connected to the jack on the bottom of the device is mounted on the user's earlobe.

On the other hand, although not shown in the block diagram of the internal device of FIG. 3, an EEG measurement program for controlling the overall EEG measurement according to the present invention and performing a process for providing an EEG measurement process and result display interface screen on a smartphone display screen may be stored in a separate storage unit (not shown) included in the control unit (not shown) of the main body or may be mounted in a separate storage unit (or memory). Of course, the EEG measurement program is a separate program from the operation program for performing the breathing meditation induction process, such as providing content according to the user's selection during the existing breathing meditation induction.

In the present invention, in order to provide the content according to the EEG measurement result, the content is provided through a computer (server) located at a remote location. However, the present invention is not limited thereto, and in FIG. 3, a plurality of contents are stored in the memory device according to the memory capacity, and the contents according to the EEG measurement result may be directly provided by the psychotherapy device itself by the user's selection.

In the present invention, the EEG measurement result may be directly stored in the smartphone terminal storage unit, or may be selectively transmitted to a remote computer server for storage management.

The processing operation of the interface screen in the present invention may be possible by touch or voice.

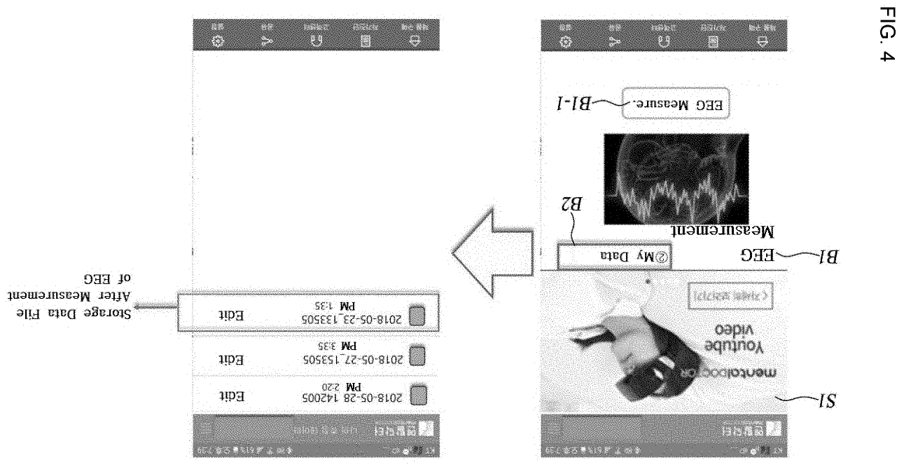

FIG. 4 is a diagram illustrating an example of an interface screen displayed on a personal terminal in a psychotherapy system using a psychotherapy device that is connected to a personal terminal and can display brain waves through Bluetooth communication according to the present invention.

Referring to FIG. 4, a screen capable of EEG measurement is displayed on the initial screen on the mobile smartphone screen S1. A button for EEG measurement (B1) and my measurement data selection button (B2) are provided. The user can measure the EEG by clicking the EEG measurement button (B1-1).

When the My Measurement Data selection button (B2) is clicked, the My Measurement Data list is displayed by date. 5 is a diagram illustrating an example of a pop-up window displayed when my EEG data file is touched in FIG. 4.

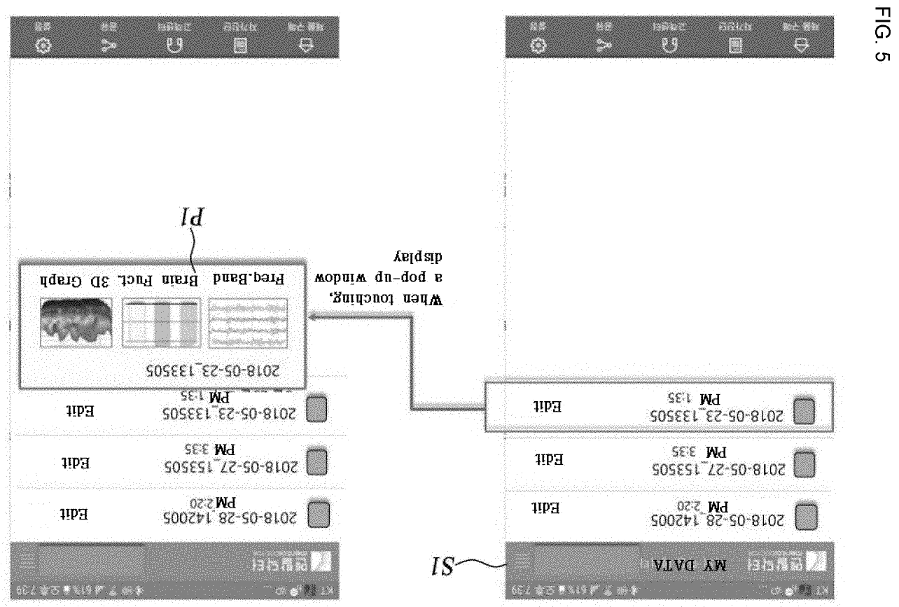

Referring to FIG. 5, when a user selects or touches a specific item from the My Measurement Data list, a pop-up window P1 is displayed. In the pop-up window, frequency band, brain function (graph), and 3D (three-dimensional) graph are displayed.

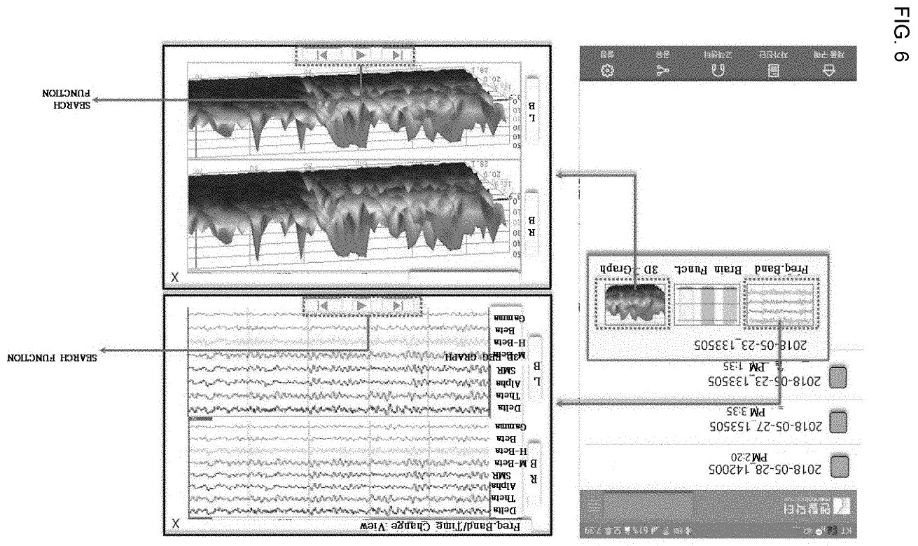

FIG. 6 is a view showing an example of a screen and a 3D (three-dimensional) graph screen after clicking a frequency band in the pop-up window displayed in FIG. 5.

Referring to FIG. 6, as in FIG. 5, the user can click (or touch) the frequency band in the My Measurement Data pop-up window to view the right brain and left brain frequency bands on a large screen. A search function button is also provided. In addition, the 3D EEG graph can be viewed for the right brain and the left brain, respectively.

7 is a graph showing changes in brain index displayed when a brain function is selected in the pop-up window displayed in FIG. 5.

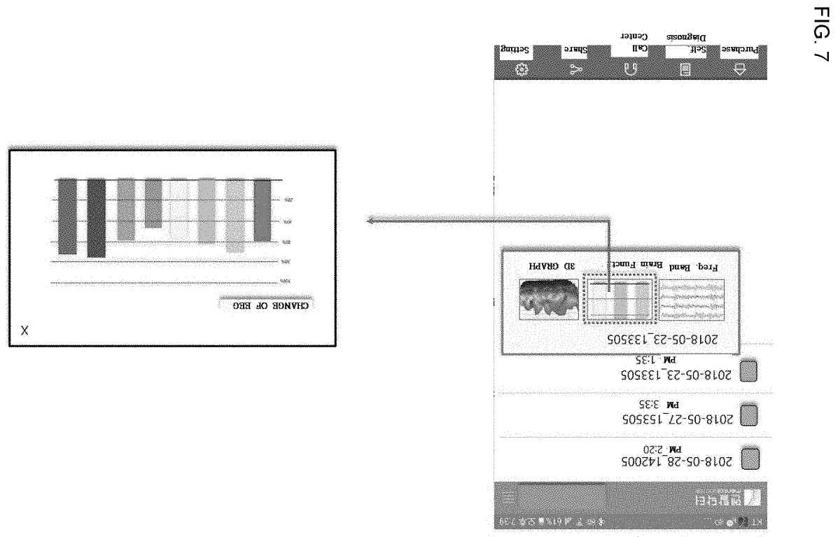

Referring to FIG. 7, as in FIG. 5, the user can select a brain function item in the My Measurement Data pop-up window to view the change in the brain index.

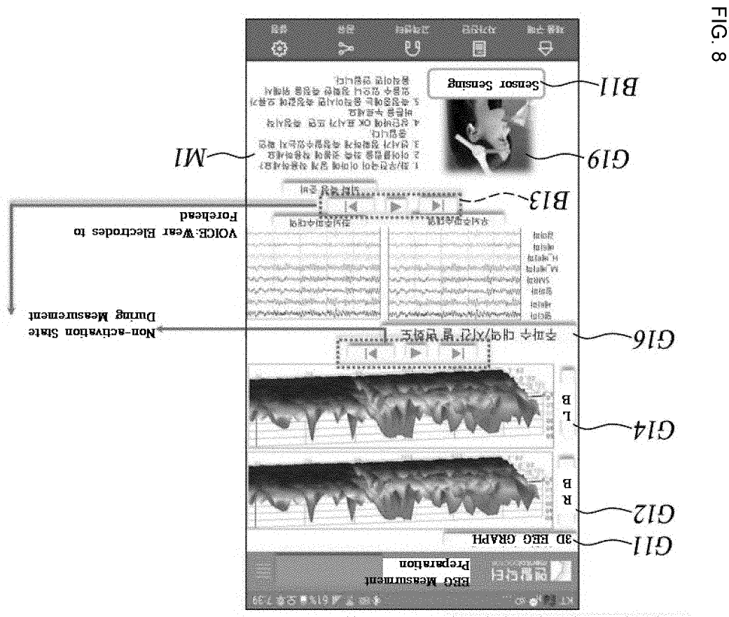

FIG. 8 is an interface screen illustrating an example of an EEG preparation screen displayed when an EEG measurement is selected on the screen of FIG. 4.

Referring to FIG. 8, when the user selects EEG measurement preparation in the communication state of the personal terminal and the headphone combined breathing meditation induction device connected to each other through Bluetooth communication as shown in FIG. 1, the EEG measurement preparation interface screen is displayed. That is, the three-dimensional EEG graph (G11) is displayed on the right brain (G12) and the left brain (G14), respectively, and the displayed graph can be played left and right by the play button (B12) (inactive during measurement).

In addition, although the degree of change (G16) for each frequency band time is displayed, there are various items. (ie, delta wave, theta wave, alpha wave, SMR wave, M_beta wave, H_beta wave, beta wave, gamma wave are displayed). It is divided into right brain frequency band and left brain frequency band, respectively. Of course, a playback area change display button B13 is provided.

As the types of the displayed EEG, delta wave, theta wave, alpha wave, SMR wave, M_beta wave, H_beta wave, beta wave, and gamma wave are generally well known in the prior art data for their characteristics and frequencies, in the present invention, the first characteristic is that such an EEG display is acquired through a sensor and displayed for each frequency as well as displayed as a brain index graph corresponding to each EEG. Of course, at this time, a brain index graph display module (program) corresponding to each EEG data is provided.

In addition, in the present invention, the brain index graph item can be additionally displayed. In this case, based on the EEG data obtained through the EEG signal measurement, the type of the EEG display can be enlarged and displayed. Of course, it can be simplified and reduced to several and displayed in the form of a report. The user may select a specific item in the initial stage of EEG measurement and output the brain index graph. In this case, the present result data may be displayed in comparison with the past data, or the present result data may be displayed compared to the average or normal data.

In addition, the wearing guidance screen (G19) of the breathing meditation inducing device combined with headphones for brain wave measurement is displayed, and the current state guidance message display button window (B11) is displayed (the message is changed and displayed, that is, the EEG sensor is being monitored or measurement is started. Or the measurement completion is displayed), the EEG measurement preparation guide is displayed. At the same time, the voice is displayed. In other words, the message “wear the ear clip (that is, the part provided with the grounding electrode for EEG measurement of the breathing meditation inducing device combined with headphones) on the earlobe” is output. After that, the message “EEG signal is being detected” is output. After that, “(If the sensor status is OK, the sensor status is displayed with the antenna indicator on the top right of the interface screen. If the number of antennas is small, check the contact again and then continue measurement) “EEG signal Good.” is output. After that, click or touch the information button window (B11) in the state where the measurement start is displayed. As a result, the EEG measurement starts.

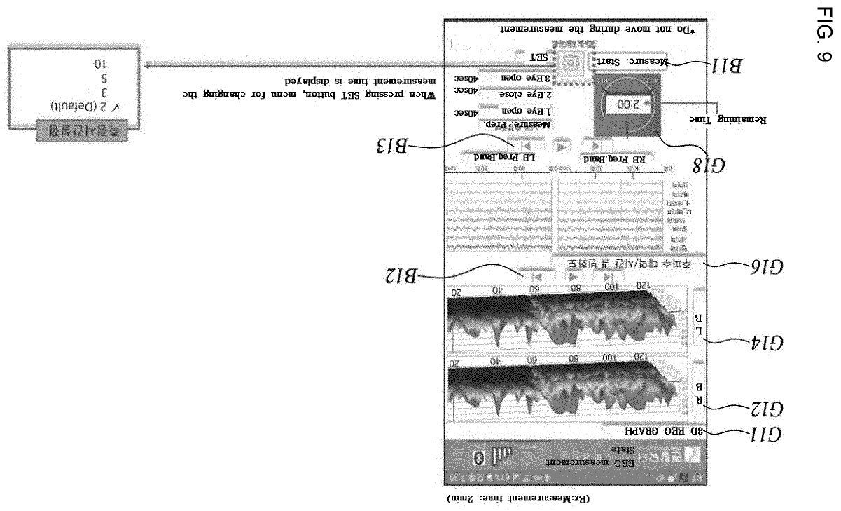

FIG. 9 is an example of a screen where EEG measurement starts when the user contacts the EEG sensor of the breathing meditation inducing device combined with headphones in FIG. 8 and touches the reference signal frequency measuring unit (ground) to the earlobe is a diagram showing

Referring to FIG. 9, for example, when the EEG measurement time is set to 2 minutes, the set time is displayed on the display unit G18. During this time, brain waves are measured. If you move during measurement, an error may occur, so do not move for accurate measurement.

EEG measurement proceeds in three steps: open eye measurement (40 seconds), closed eye measurement (40 seconds), and open eye measurement (40 seconds). However, it is not limited thereto. On the other hand, it is also possible to measure 40 seconds for each specific mode of the breathing meditation induction device combined with headphones of the present invention, and the measurement time can be changed by clicking the setting button (3, 5, 10 minutes).

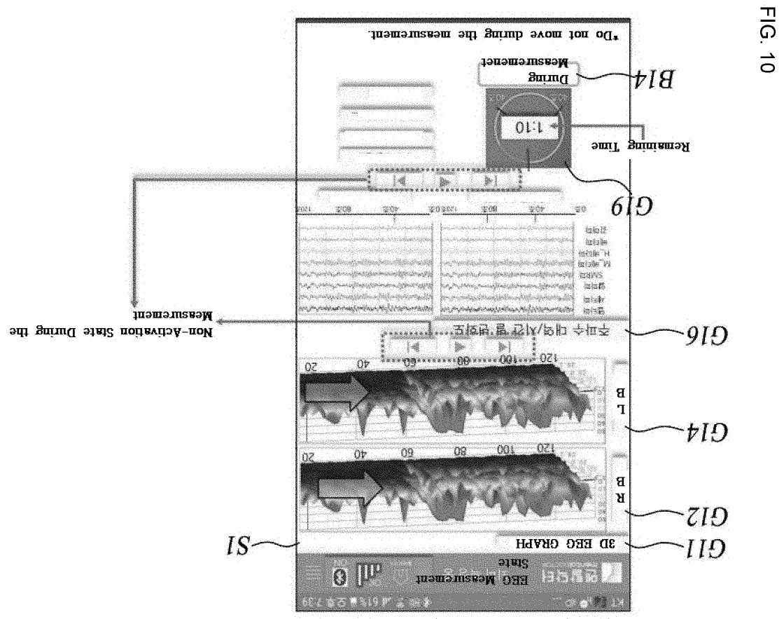

FIG. 10 is a view similar to FIG. 9 as showing an interface screen in which EEG measurement is continuing in FIG. 8.

Referring to FIG. 10, the remaining measurement time is displayed on the display unit G18. During measurement, the EEG graph and frequency band display button are displayed inactive.

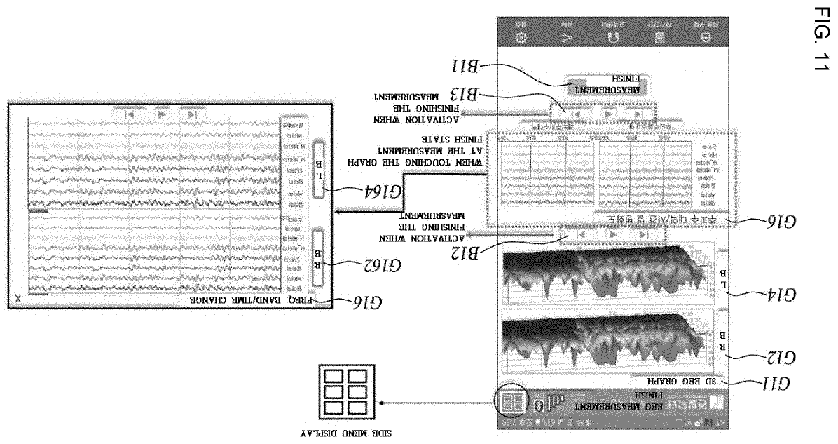

FIG. 11 is a view showing an example of the EEG measurement completion screen in which the EEG measurement in FIG. 8 is completed.

Referring to FIG. 11, it shows a state of completion of EEG measurement. When the measurement is completed, the play buttons B12 and B13 are activated. When the measurement complete button (B11) is selected, it moves to the measurement completion result page. At the top right of the interface screen, a side menu is displayed, that is, various functions such as display of comparison data or sharing can be selected.

When the measurement is completed, the degree of change by frequency band/time can be enlarged and displayed.

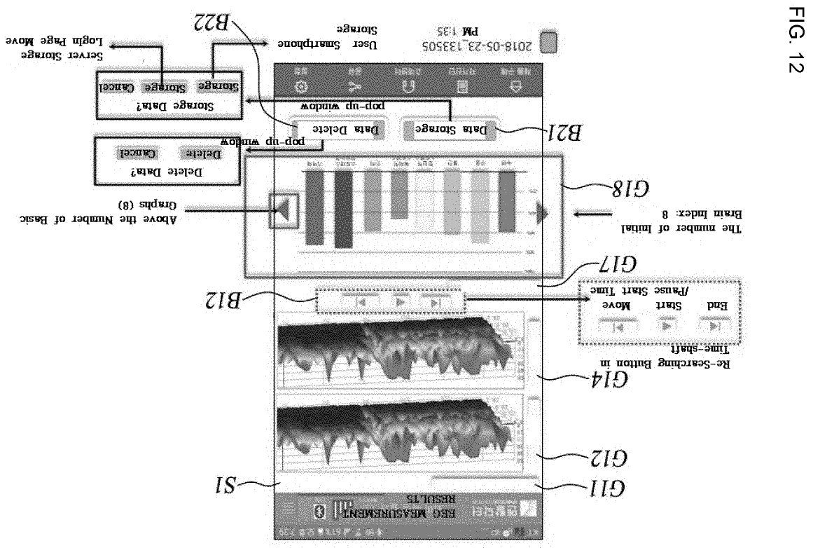

FIG. 12 is a view showing an example of the EEG measurement result screen in which the EEG measurement in FIG. 8 is completed.

Referring to FIG. 12, when the measurement complete button is selected, a 3D graph and a graph of changes in brain index may be displayed. The initial number of brain index graphs is 8, but is not limited thereto. When adding, it can be displayed by extending it to the left and right. A data save button (B21) and a data delete (B22) button are provided at the bottom of the brain index change graph. Data can be stored in the smartphone memory or on a remote server (of course, a login process is required).

In relation to the brain index graph, various contents corresponding thereto are stored in a remote computer (server) and provided according to the user's request. can be played back. After playback, the brain waves are sensed and measured again, and the data can be compared with that before content playback.

In other words, the user is connected to a personal terminal with a built-in breathing meditation induction device for brain wave measurement and a program capable of displaying and storing EEG signals via Bluetooth and the personal terminal is connected to a computer server for storing EEG signals.

By operating the application of the personal terminal, measuring the EEG signal, and grasping one's own state through the brain index measurement bar graph, the brain index measurement bar graph is about the user's mental state after using the headset and breathing meditation induction device Each type is divided into various items and displayed, and when the item is selected by touch or voice, one or more contents provided in advance are downloaded through the computer (server), and the content can be played by selecting a specific content by touch or voice.

In the present invention, in the state of not measuring EEG, by selecting a self-diagnosis item (ie, questionnaire) with water on the interface screen provided by the operating program of the present invention, the result of one's current mental state can be scored and viewed, and the A self-diagnostic item-based content provision service that allows content matching the score to be played can also be provided.

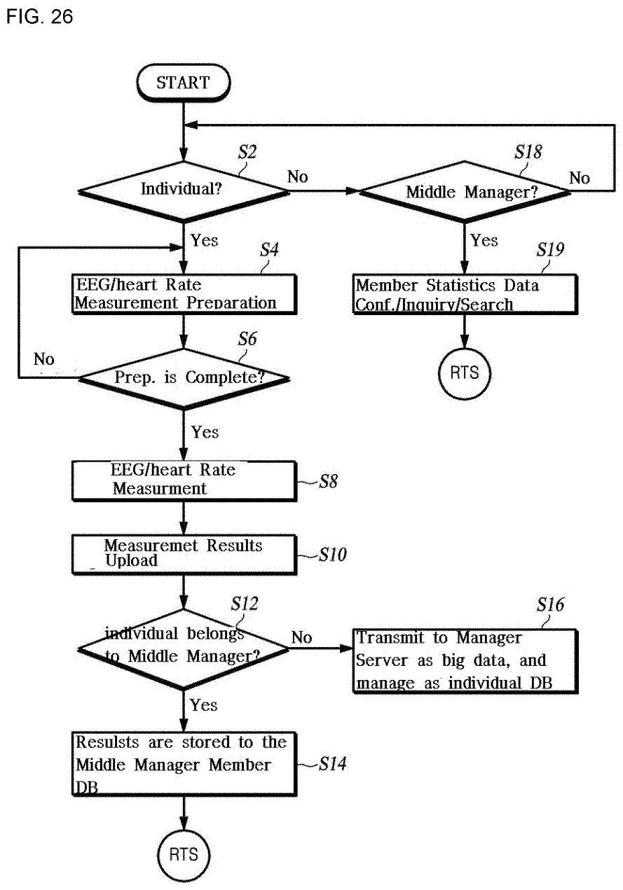

Alternatively, you can directly measure the EEG to obtain a brain index graph according to the measurement result and view the scored results at the same time, and select a specific item corresponding to the corresponding brain index graph (for example, you can select a graph with a low item index) If selected), the corresponding content is provided, and the user can play the content to improve the brain index graph of the corresponding item based on the brain index. This may be similar to providing content through the result of the self-diagnosis item. However, this is a completely different method from the method according to the self-diagnosis item in that it provides an IQ graph based on EEG measurement and then provides various functions according to the selection of the IQ graph, and is a useful method characteristically proposed in the present invention. method. Of course, it will be possible to provide a detailed description of the brain index graph to the user in the form of a report (see FIG. 26).

13 is a diagram schematically illustrating a process of storing data in a server and a personal terminal when the data storage button is selected in FIG. 12.

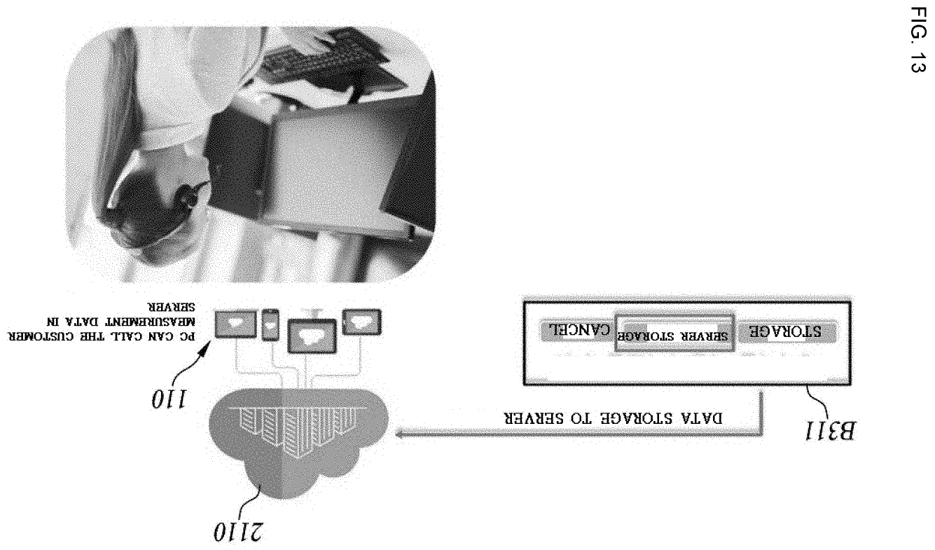

Referring to FIG. 13, similarly to FIG. 12, measurement result data may be stored in a remote computer (server) 2110 under the selection of the button unit B311, and in the PC 110, the computer (server) (2110) in the customer measurement data can be called. Through the PC 110, the customer's past data may be provided through a call center consultation, remote consultation through the Internet, or real-time online consultation, and other services will be provided in response to various customer requirements.

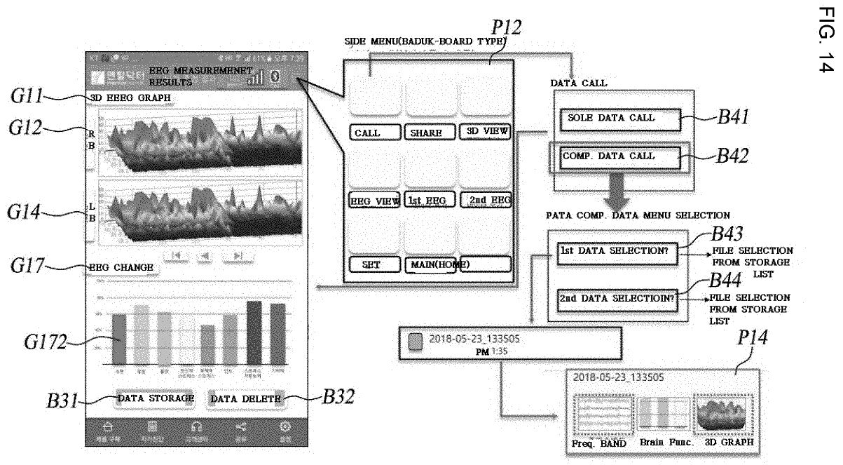

FIG. 14 is a detailed explanatory diagram of a side menu in the EEG measurement result screen of FIG. 12.

Referring to FIG. 14, the side menu pop-up window (P12) has provided the import, share, two 3D graph views, two brain index graph views, the first EEG data view, the second EEG data view, settings, and main (home) buttons.

When data import is selected, single data import (B41) comparative data load (B42) is displayed, and if later comparative data import is selected, select a file from the save list depending on whether the first data selection or the second data selection is made. indicate In the selected data, the frequency band, brain function (brain index graph data), and 3D graph can be viewed through the pop-up window (P14).

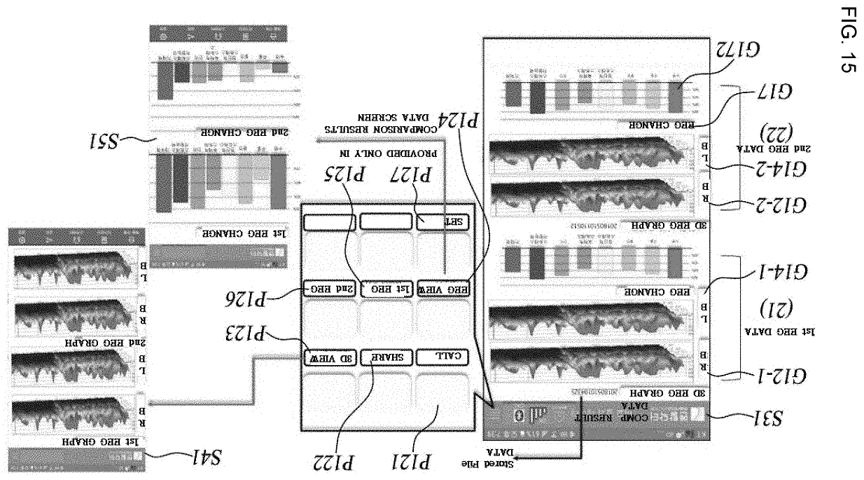

FIG. 15 is a diagram illustrating an example of a comparison result data screen in the EEG measurement result screen of FIG. 12.

Referring to FIG. 15, a comparison result data display interface screen can be viewed. That is, the 3D EEG graph can be viewed by subdividing the first and second left brain and right brain. Also, you can select and view data on a specific screen by enlarging the side menu.

Namely, call (P121), share (P122), two three-dimensional graph view (P123), two brain index graph view (P124), first EEG data view (P125), second EEG data view (P126), setting (P127), a main (home) button (P128) are provided. This side menu is provided only on the comparison result data screen. You can see the enlarged screen or graph data by clicking each button.

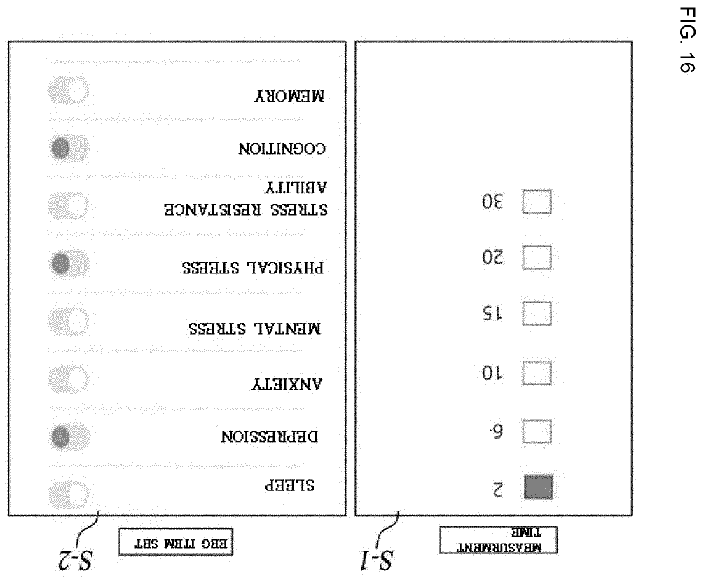

FIG. 16 is an interface screen for setting brain index measurement items displayed on a personal terminal in a breathing meditation induction system using a breathing meditation induction device combined with headphones that can display brain waves by being connected through Bluetooth communication with a personal terminal according to the present invention. It is a drawing showing an example.

Referring to FIG. 16, the user sets the brain index items and selects the desired item from among sleep, depression, anxiety, mental stress, physical stress, stress resistance ability, cognition, and memory). The measurement time is also selected from among 2 minutes, 6 minutes, 10 minutes, 15 minutes, 20 minutes, and 30 minutes (2 minutes is selected). It is assumed that each brain index is pre-arranged so that data corresponding to the measured value is output according to a preset database.

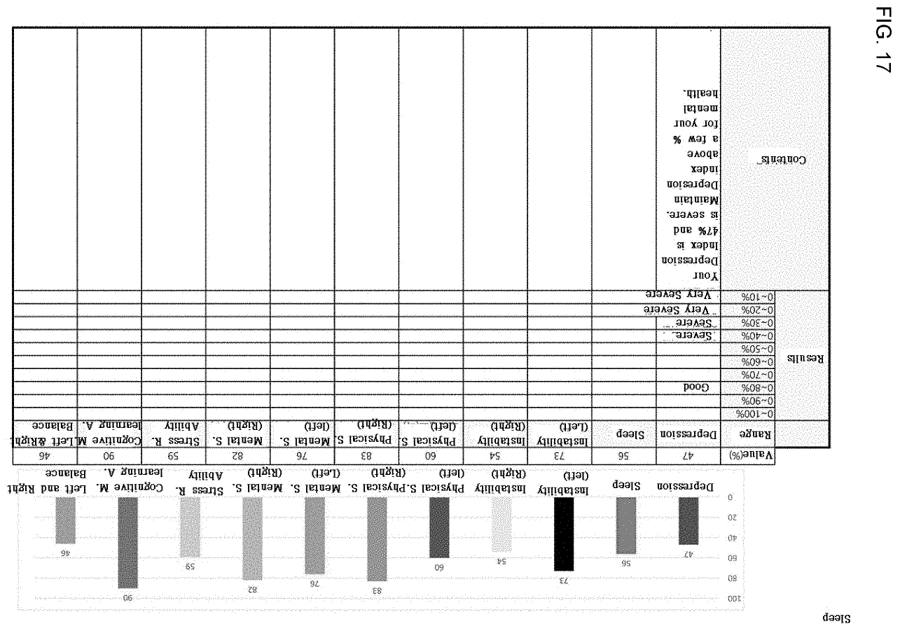

FIG. 17 shows an example of a more specific brain index display interface screen displayed on a personal terminal in a psychotherapy system using a breathing meditation inducing dev II-Tice combined with headphones that can display brain waves by being connected through Bluetooth communication with a personal terminal according to the present invention.

Referring to FIG. 17, for example, the brain index measurement data is analyzed for depression, sleep, instability (left), instability (right), physical stress (left), physical stress (right), mental stress (left), mental stress (right), stress resistance ability, cognitive memory learning ability, and left and right brain balance are set and measured (Each graph is assumed to measure EEG and output matched data through a preset database.).

Measurement result, analysis value (%) is, for example, depression 47, sleep 56, instability (left) 73, instability (right) 54, physical stress (left) 60, physical stress (right) 83, mental stress (left) 76, mental stress (right) 82, stress resistance ability 59, cognitive memory learning ability 90, left and right brain balance items were measured as 46 items. As a result of division, the measured values can be divided at 10% intervals. Depression of 50% or less is judged as severe, and less than 20% is considered as very severe. A score of 80% or higher is judged to be good for the depression index. The same index applies to other items as well. In the remarks column, along with the opinion of the evaluation result of the current depression index, the opinion that maintaining the depression index above a few percent is helpful for mental health. This opinion is the same for other items. On the other hand, this item is only an example and can be applied differently depending on the program setter, of course.

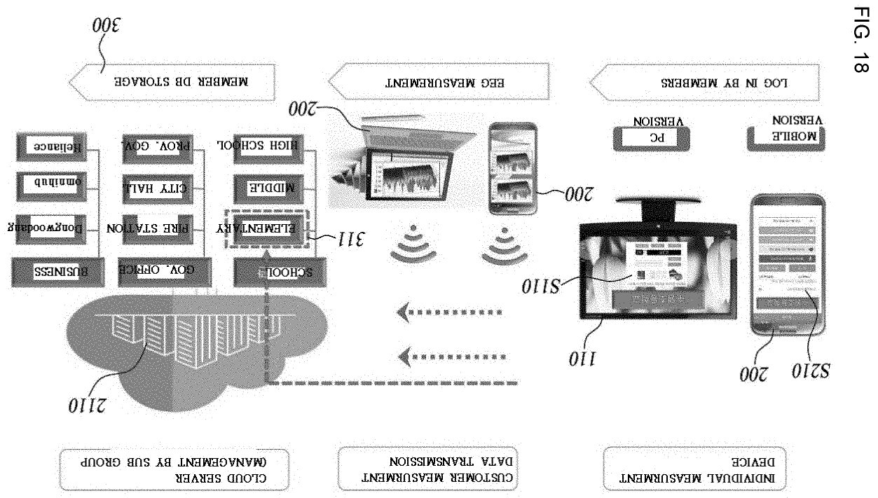

FIG. 18 is a conceptual diagram explaining the process for saving EEG measurement results to a cloud database: the results measured by a personal measurement device in a breathing meditation induction system using a breathing meditation induction device combined with headphones that can display brain waves by being connected through Bluetooth communication with a personal terminal according to the present invention.

Referring to FIG. 18, it is possible to access a cloud server (referred to as an operator computer server) 2110 through a communication network through a mobile terminal 200 or a personal PC terminal 110 as a personal measurement device. It is possible to access the database 300 by inputting an ID and password through the login screens S210 and S110, respectively, through the mobile terminal 200 or the personal PC terminal 110, which is a personal measurement device.

In addition, when measuring EEG, it is not shown through the mobile terminal 200 or personal PC terminal 110, which is a personal measuring device, but measures EEG while connected to the headphone and breathing meditation inducing device 10 of FIG. The EEG measurement result data may be transmitted and stored in the mobile terminal 200 or the personal PC terminal 110 or the cloud computer server 2110.

The cloud server 2110 is connected to the database 300, and the database 300 is divided into a plurality of subgroups, and a plurality of small groups are divided into the subgroups to store and manage data.

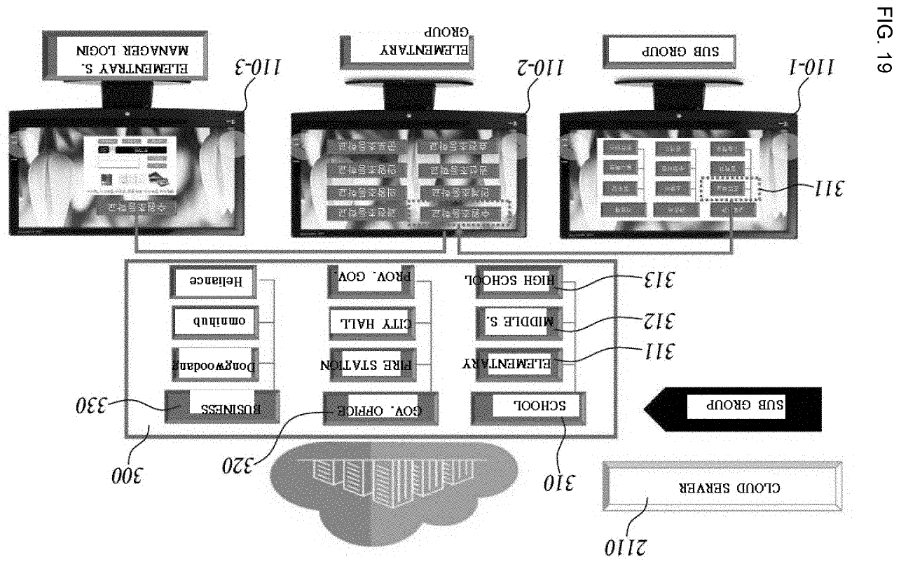

FIG. 19 is a conceptual diagram schematically illustrating a process of accessing a database of a cloud server through a personal terminal connected to the cloud server through a communication network in FIG. 18.

Referring to FIG. 19, as described in FIG. 18, a plurality of subgroups are provided in the database 300 of the cloud computer server 2110. For example, an educational institution 310, a government office 320, and a corporation 330 are provided, respectively, an elementary school 311, a middle school 312, and a high school 313 are provided, and also a fire station, Suwon City Hall, Gyeonggi-do is provided, and companies such as Dongwoodang, Omnihub, and Heliance are provided (for example, this is indicated and a database of various small groups may be provided).

For example, if you log in to the elementary school 311 and want to view data, you can log in from a personal PC terminal and visit subgroups and small groups.

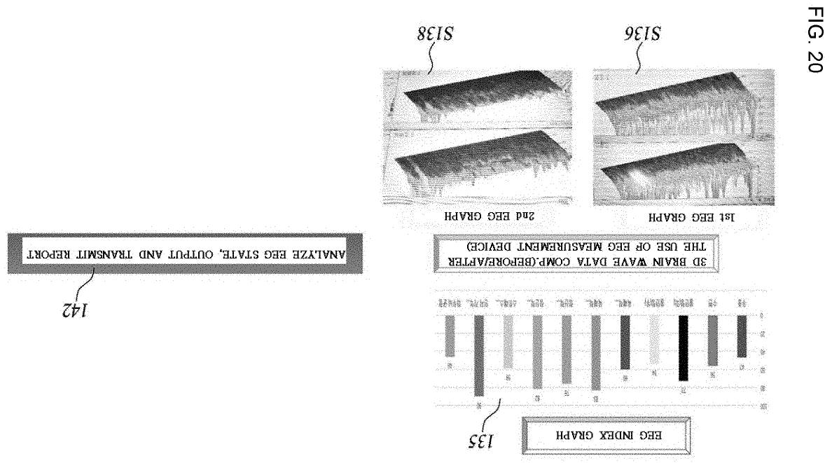

FIG. 20 is a brain index graph and three-dimensional brain wave data before and after use provided in a breathing meditation induction system using a breathing meditation induction device combined with headphones that can display brain waves by being connected through Bluetooth communication with a personal terminal according to the present invention; It is a conceptual diagram schematically showing the process of analyzing the brain index state and outputting it as a reporter.

Referring to FIG. 20, in the present invention, the brain wave of the user is measured through the brain wave measurement sensor of the breathing meditation inducing device combined with headphones to represent the brain index graph (S135). The three-dimensional EEG data can be compared through the screen before use (S136) and the screen after use (S138), and the step (S142) of outputting and transmitting the report by analyzing the brain index state is performed, at this time items shown in the report data screen (not shown) may be, for example, mental distraction and stress state, behavioral direction, emotional direction, left and right brain balance, and self-feedback ability. However, the contents of the report data screen are not limited to these contents, and the graph items may be implemented through the report function of the EEG program 2100 based on the EEG measurement result data. Users can compare their EEG results with past data and view them. Alternatively, you can compare and view the graph of your result data compared to the average or normal graph. When the graph of the G135 is clicked, one or more contents corresponding to the corresponding measurement graph are displayed, and it is possible to execute the contents by clicking the corresponding contents. The corresponding contents are prepared in advance by the operator in a database according to the measurement results. Although already described above, the brain index graph can be displayed corresponding to the measured EEG frequency data, and the displayed data values are converted into a graph to be displayed, and the corresponding EEG graph is the measured EEG frequency data. It is of course possible that a new addition can be made by combining them.

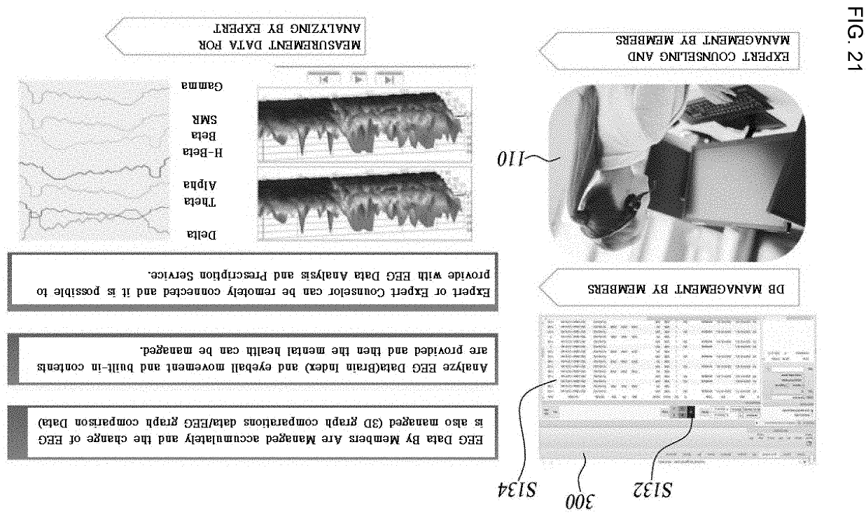

FIG. 21 is a personal terminal and a personal terminal according to the present invention connected through Bluetooth communication in the breathing meditation inducing system using a headphone and breathing meditation inducing device capable of displaying brain waves in the state of accessing the cloud server through a personal terminal, database for each member It is a conceptual diagram showing the state to be managed and displayed.

Referring to FIG. 21, since EEG data according to the present invention is stored and managed in the cloud server, the user requests through the operator or manager from a remote location, and the operator or manager of the cloud computer server 2110 through a personal PC terminal. By accessing the database 300, specific individual EEG measurement data can be viewed through the score display screen (S132) and the EEG date screen (S134). In addition, the user can receive various services through the operator or administrator through the login process. For example, since EEG test data for each member is accumulated and managed to manage EEG state change, you can view 3D graph comparison data and brain index graph comparison data at any time. In addition, users can receive eye movement and customized contents by analyzing EEG data (brain index), making it possible to manage their mental health through their breathing meditation induction process.

In addition, it will be possible to receive EEG data analysis and prescription services by connecting remotely through an expert or professional counselor.

On the other hand, although not shown, the data recorded in the cloud server using the device according to the present invention may be used as big data as various analysis data.

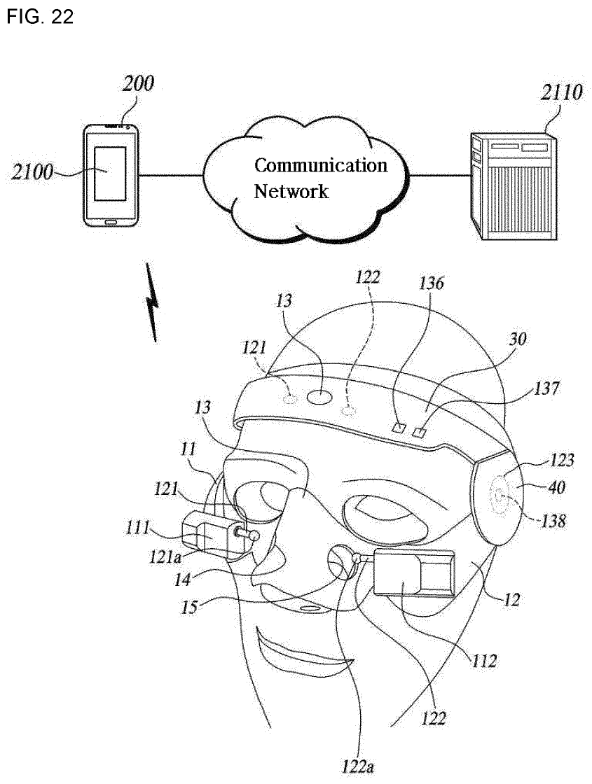

FIG. 22 is a breathing meditation inducing device that can measure actual brain waves in a breathing meditation inducing system using a headphone and breathing meditation inducing device that can display brain waves by being connected through Bluetooth communication with a personal terminal according to the present invention. It is a figure which shows an example of a state.

Referring to FIG. 22, the sensors 121 and 122 for EEG measurement are installed with the inner side (part in contact with the forehead) attached to the headphone 30 of the breathing meditation inducing device 10 combined with a headphone that can measure EEG, and both sides are extended extensions It is electrically connected to the control unit (not shown) through the unit. After the user contacts the EEG sensors 121 and 122 on their forehead, the headphones 30 of the breathing meditation induction device 10 capable of measuring EEG A portion of the ground sensor 123 (removing noise generated during EEG measurement and generating a reference frequency compared to EEG) located in the ear cover 40 SO of the lobe is brought into contact with the user's left earlobe. This completes the preparation for EEG measurement. The EEG measurement setting can be performed by executing the EEG measurement program 2100 of the personal terminal 200 connected through Bluetooth (however, it is not limited thereto, and the EEG can also be performed in the headset and breathing meditation induction device 10 capable of EEG measurement).

The measurement program 29 may be installable After the EEG measurement is completed, the result data is transmitted to the personal terminal 200 through Bluetooth, and optionally transmitted to the remote computer server 2110 to be stored and managed. will of course be possible.

In the drawing, there is a sensor that can check the electrocardiogram in the headphone part, and the pulse rate (heart rate) (pulse rate (heart rate) sensor to be described later) (not shown) and oxygen saturation (oxygen saturation measuring sensor to be described later) can be measured by touching a finger. It is of course possible that the sensing unit may be additionally configured.

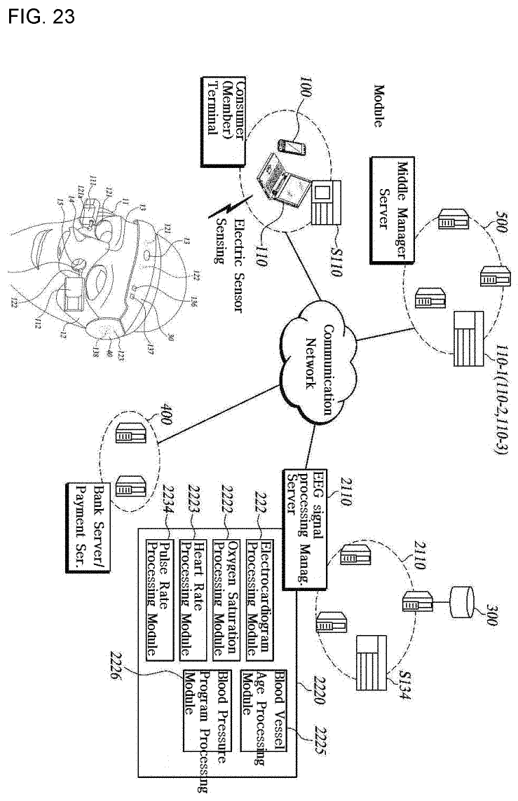

FIG. 23 is a diagram showing an example of a management platform system of EEG measurement signal big data through an individual and an intermediate manager in a breathing meditation induction system using a breathing meditation induction device combined with headphones that can display brain waves by being connected through Bluetooth communication with a personal terminal according to the present invention.

Referring to FIG. 23, the management platform system of EEG measurement signal big data through individuals and middle managers using the headphone and breathing meditation induction device, according to the present invention, includes an individual or a consumer (member) smartphone terminal or PC terminal, a middle manager server 500 and, EEG measurement signal processing system manager server 2110 and a bank server and/or a billing server 400.

The individual or consumer (member) smartphone terminal 200 or PC terminal 110 is connected to the headphone and breathing meditation inducing device 10 equipped with an EEG sensor through Bluetooth communication. The PC terminal 110 or the smartphone terminal 200 may execute various functions, for example, EEG measurement, heart rate measurement, pulse measurement, blood sugar measurement, oxygen saturation measurement, and the like through the interface screen S110. Of course, this measurement is performed through the EEG signal sensor, and other heart rate measurement, pulse measurement, or oxygen saturation measurement (performed through the heart rate/pulse sensor 136 and oxygen saturation sensor 137). In the present invention, a sensing unit on the upper surface of the device (13) was installed. Blood vessel age measurement is processed by synthesizing the results measured by the electrocardiogram sensor 138 and other oxygen saturation measuring sensors 137. The sensors may be installed in the headphone combined breathing meditation inducing device 10 or installed in the headphone unit of the headphone combined breathing meditation inducing device 10, or an LED device may be installed in the headphone combined breathing meditation inducing device 10 separately. Therefore, it will be possible to emit LED light to the body of the measurer, and to measure heart rate, pulse rate, blood glucose, and oxygen saturation through the received radiated light. In the present invention, the process of sensing a signal may employ a previously developed sensing device (not shown), and then processing the measured signal through an operation program in the control unit of the headphone and breathing meditation induction device 10 It will be possible. In addition, if the measurement signal is uploaded through the breathing meditation induction device 10 combined with headphones, it will be processed by the EEG measurement signal processing system manager server 2110 and provided so that the processing result can be downloaded.

Various devices are provided in the program storage unit 2220. Unexplained reference numeral 222 denotes an electrocardiogram processing module that processes and displays an electrocardiogram, and unexplained reference numeral 2222 denotes an oxygen saturation processing module that measures and processes oxygen saturation, Unexplained reference numeral 2223 denotes a heart rate processing module for processing a heart rate, unexplained reference numeral 2224 denotes a pulse rate processing module for processing and displaying a pulse rate, and unexplained reference numeral 2225 denotes a blood vessel age processing module for determining and displaying blood vessel age, and reference numeral 2226 denotes a blood pressure processing program module that displays and processes blood pressure by determining the measured electrocardiogram, oxygen saturation, and heart rate.

The blood vessel age and blood pressure measurement are processed by the program, and the ECG, pulse rate, oxygen saturation, and heart rate programs are also processed. The program for processing electrocardiogram, pulse rate, oxygen saturation, etc. generally uses the functions of well-known devices. In the present invention, the sensor is used to process the measured result value through the program, and provides a 2D or 3D display function.

The middle manager server 500 includes other middle manager servers 510, 520, and 530 divided by a plurality of job types, and the middle manager server stores information about a plurality of individuals and manages this information. The middle manager server 500 may execute various functions, for example, statistical processing for each member through the interface screens S110-1, S110-2, and S110-3.

The EEG measurement signal processing system manager server 2110 functions as a cloud server, and the signal measured through the headphone and breathing meditation induction device 10 is stored through a personal terminal or a smart phone terminal, and then the stored measurement data as well as the individual It may be viewed through the interface screen of the terminal or smart phone terminal, and may be uploaded to the EEG measurement signal processing system manager server 2110 connected through a network communication network simultaneously or sequentially with storage. The uploaded data is divided into the database 300 and stored and managed in separate databases 310, 320, and 330. The manager server 2110 may display a service function through the interface screen S14. In addition, the manager server 2110 may statistically classify and display information on heart rate, pulse rate, oxygen saturation, glycemic index, blood vessel age, etc. as well as related statistical screens related to EEG signal processing.

The middle manager server 500 can be classified by various groups, and serves as a manager who manages schools, government offices, private institutes, insurance companies, as well as numerous individuals, manages individual data for each group, and can extract statistical data. And, it is possible to utilize the measured data, and through this, it is possible to separately register as an intermediate manager in the EEG measurement signal processing system manager server 2110, pay for the cost, and then receive a separate service. Middle managers can individually manage measurement information for individuals in a specific group.

According to the present invention, individuals download and install an EEG signal measurement program through a personal terminal or a PC terminal, and then connect to an EEG signal (not limited thereto, pulse rate, heart rate, oxygen saturation can be measured) through a Bluetooth communication connection with the breathing meditation inducing device 10 combined with headphones. The measurement result can be viewed on a graphic screen, and historical data and current data can be compared. You may also see statistical data. In addition, the measured EEG signal may be uploaded in real time to the EEG signal processing system manager server to be managed as big data except for personal information.

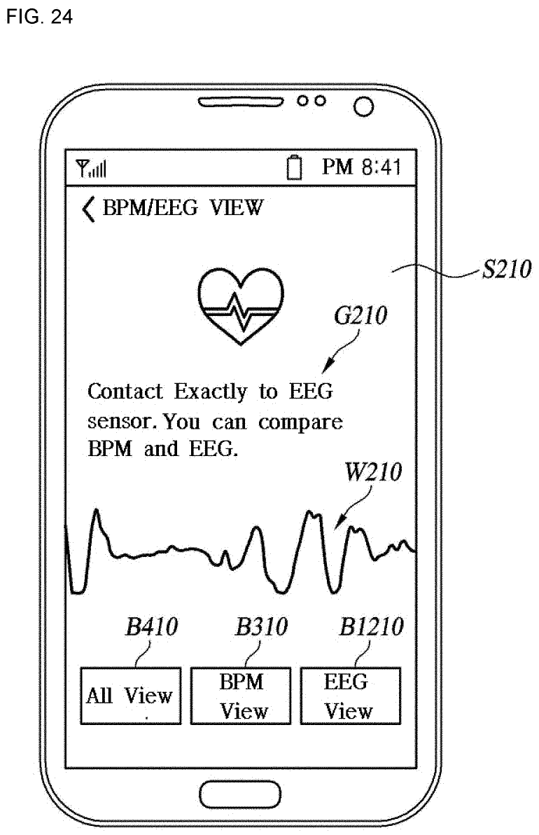

FIG. 24 is a diagram illustrating an example of a screen for performing an EEG signal and a heart rate measurement result in the personal terminal of FIG. 23.

FIG. 24, as an example of the EEG signal processing result screen in the personal terminal is displayed, the heart rate/EEG measurement index view function is displayed on the screen S210, and the EEG measurement is performed through the message display screen G210. After the measurement, the related data can be displayed through the waveform screen W210 (similar to the waveform data of the EEG measurement result). Simultaneous view of function buttons on the screen (B410), heart rate view (B310), and EEG measurement index view (B1210) are exemplified.

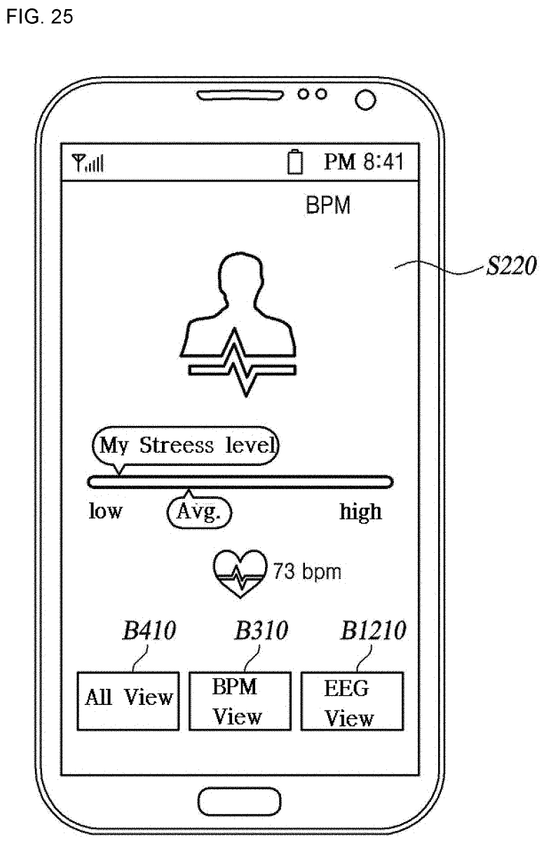

FIG. 25 is a diagram illustrating an example of a screen that displays an index such as a stress index while performing an EEG signal and a heart rate measurement result in the personal terminal of FIG. 23.

FIG. 25, as an example of the EEG signal processing result screen in the personal terminal is displayed, the heart rate (BPM)/EEG index viewing function is displayed on another (interface) screen S220, and related graphics and Index data is shown on the screen. Simultaneous view of function buttons on the screen (B410), heart rate view (B310), and EEG measurement index view (B1210) are exemplified.

FIG. 26 is a flowchart illustrating an example of a process of measuring an EEG signal and a heart rate in an individual and/or middle manager terminal in FIG. 23.

Referring to FIG. 26, in the EEG signal processing management system according to the present invention, when an individual logs in (S2), (the individual inputs ID and PW on the initial web page of the management system operation (or by fingerprint recognition, etc.) If you log in or download the EEG signal processing program from your personal terminal and run it, it will be connected to the EEG signal processing management system (or middle manager server) and then, if you are an individual, EEG/heart rate (or oxygen saturation, Or blood vessel age measurement, etc.) prepares for measurement (S4), determines whether the state is ready (S6), and the determination of the ready state can be determined by looking at the network connection state between devices and the program operation state.

Thereafter, EEG/heart rate measurement is performed through the breathing meditation inducing device 10 combined with headphones (S8), and thereafter, EEG/heart rate measurement result data is stored and uploaded (S10).

At this time, when uploading, data is stored and managed in the manager server, and personal information is excluded.

Thereafter, it is determined whether the individual belongs to the middle manager (S12). If the individual is an individual belonging to the middle manager, the result data is stored in the middle manager member database (S14). 27 is a flowchart illustrating an example of a login process for performing EEG signal and heart rate measurement in the middle manager terminal in FIG. 23.

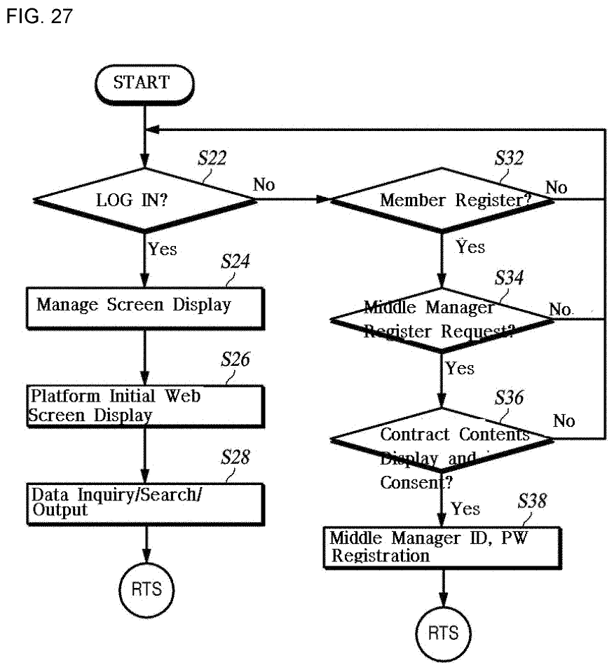

Referring to FIG. 27, in general, when an individual tries to measure EEG/heart rate through the breathing meditation inducing device 10 combined with headphones, the program is operated in the personal terminal or PC terminal, and the ready state for EEG measurement is In the completed state (network connection state), if an individual belonging to the middle manager logs in with the middle manager ID and PW, the manager screen is displayed, and the EEG measurement program (actually a program that can perform various additional functions in addition to EEG measurement) When operating, the name is limited for convenience), after the login (S22) process, the platform (referring to a web environment for measurement, management, or inquiry of brain waves and various data, etc., but is not limited thereto) initial web screen (not shown) is displayed (S24), and then the manager screen is displayed (S26), and functions such as data inquiry/search/output can be selected on the manager screen (S28).

If the login is not performed, a member registration step (S32) is performed, and a middle manager registration request (S34) is performed, which is a request process for becoming an intermediate manager.

Afterwards, the EEG measurement signal system manager server requests to display the contract details and agree to the request for middle manager registration (S36). Thereafter, through the consent of the requester, a middle manager ID is assigned and PW registration is managed (S38). 28 is a flowchart illustrating an example of a billing process after measuring an EEG signal and a heart rate in the middle manager terminal in FIG. 23.

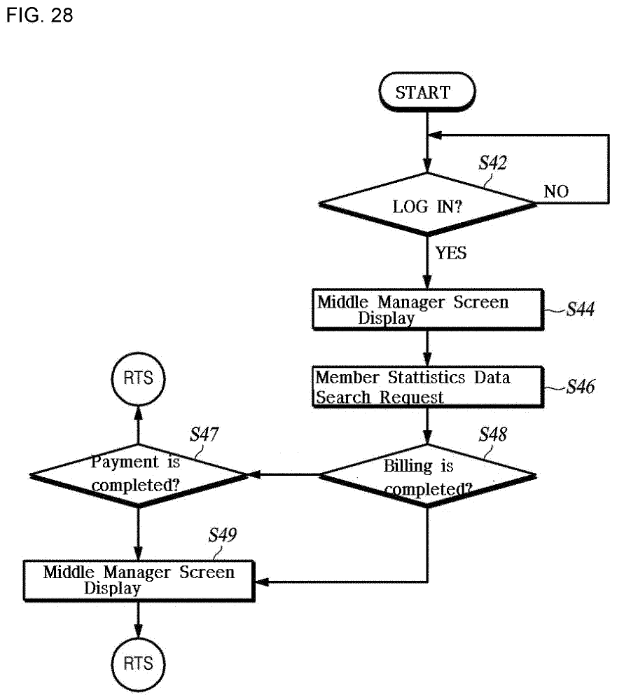

Referring to FIG. 28, when the middle manager logs in (S42), the middle manager screen is displayed (S44), and when a member statistics data search is requested (S46), it is determined whether the billing status for the middle manager is checked (S48) and charged and it is determined whether the status is completed (S47). After that, when the billing is completed, the member statistics data requested by the middle manager are displayed, and the middle manager can then manage the information about the individual he is managing by inquiring/searching/editing, etc. If the billing is not completed, the payment completion status is determined (S47), and when the payment is completed through the bank server 400 or the billing server, the service is provided.

On the other hand, although not shown, according to the present invention, a measurement signal processing program that receives and processes biological signals such as an individual's brain wave signal, heart rate, and oxygen saturation through the control unit is installed by installing other measuring sensors on the PCB in addition to the brain wave measuring sensor. did Through this, an individual can view data about his or her current state through his or her terminal or PC terminal, and can be informed of statistical data (by group) about his or her current state through an intermediate manager. In addition, through the EEG measurement signal system manager server, the statistical data collected in the big data format can be received. The EEG signal system manager server classifies the uploaded data by age, gender, and occupation, excluding personal information, receives EEG signals, oxygen saturation signals, pulse rate, and heart rate signals, classifies them as big data and provides them. In addition, it is possible to measure the age of blood vessels in the corresponding age group, and through this, it will be possible to predict diseases such as high blood pressure and diabetes. If necessary, if the biosignal measurement index for a specific individual exceeds the average and is a dangerous value, a message is sent to the individual by push mail, e-mail, or other alarm signal (for example, your EEG is a warning level, or your blood pressure index is at a warning level, or a message stating “Get your blood pressure checked immediately, get an EEG scan immediately.”).

According to the present invention, the user wears the breathing meditation induction according to the present invention on the face, detects the EEG through the sensing device for EEG sensing that is in contact with the forehead above both eyes, and only looks at the light displayed through the LED bar with the eyes. With this, it is possible to accurately and comfortably execute brain stimulation motions to see the changes in the brain waves before and after using the device as a three-dimensional waveform and in a bar graph in various ways.

In addition, by downloading the application for the breathing meditation induction system through a personal terminal capable of wired or wireless communication, using the breathing meditation induction connected through Bluetooth communication, the brain waves are detected, and the changes in the brain waves are variously converted into three-dimensional waveforms and bar graphs. By being able to sleep, mental stability and psychotherapy are possible.

In addition, the individual can promote mental and physical stability by clicking on the data obtained through the analysis result and executing the appropriate content for the data, and after executing the content (provided by the operator of the present invention), various signals such as EEG signals Since it can be measured and analyzed, the effect of improving mental and physical conditions such as mental and physical stability through the contents of the present invention can be visually confirmed.

In addition, according to the present invention, since the middle manager who manages a plurality of individuals can manage specific individuals by field, meaningful data can be collected for each group according to the ability of the middle manager, and also the manager of the present invention can manage big data can serve as a manager who can collect and provide a platform to middle managers.

### INDUSTRIAL APPLICABILITY

In addition, according to the present invention, an individual can additionally collect bio-signal-related data such as heart rate, blood vessel age, oxygen saturation, and electrocardiogram as well as brain waves, and through this, check the current state of the body through bio-signal measurement You can check your body management status by uploading it.

In the above, preferred embodiments of the present invention have been illustrated and described, but the present invention is not limited to the specific embodiments described above, and it is common in the technical field to which the present invention pertains without departing from the gist of the present invention as claimed in the claims. Various modifications may be made by those having the knowledge of, of course, and these modifications should not be individually understood from the technical spirit or perspective of the present invention.

1. A breathing mediation induction device combined with headphones for sensing brain wave signals comprising:
a body mounted on the user's face,
a control device being electrically coupled to the body, and that provides a driving signal to the body,
wherein the body includes first and second opening and closing means for opening and closing the nostril of the user's nose; first and second driving means for driving the first and second opening and closing means, respectively; and a mounting means for mounting the body on a specific part of the face,
wherein headphones are integrally coupled to the main body and include an earmuff for covering the user's ear and having a headphone installation unit for covering the user's head,
wherein a pair of EEG measuring electrodes capable of measuring EEG, which is a kind of biosignal, is installed on the inner surface of the headphone installation unit,
wherein one side of the headphone includes a sensing means for measuring EEG composed of one electrode for grounding, and includes a USB interface for coupling to a personal computer or smart phone,
wherein the A breathing mediation induction device senses an EEG signal, and is connected to a personal computer or smart phone, and transmits the sensed EEG signal to the personal computer or smart phone by operating the user's EEG according to the measurement request signal using the personal computer or smart phone.

2. A breathing mediation induction device according to claim 1, wherein the sensing means for measuring EEG is located so as to be in contact with the upper forehead of both eyes and made of electrically conductive materials is a device for inducing breathing meditation combined with headphones for sensing EEG signals.

3. A breathing mediation induction system for displaying and storing EEG signal comprising:
a body mounted on the user's face,
a control device being electrically coupled to the body, and that provides a driving signal to the body,
wherein the body includes first and second opening and closing means for opening and closing the nostril of the user's nose; first and second driving means for driving the first and second opening and closing means, respectively; and a mounting means for mounting the body on a specific part of the face,
wherein headphones are integrally coupled to the main body and include an earmuff for covering the user's ear and having a headphone installation unit for covering the user's head,
wherein in an application through a personal terminal, when the user terminal selects the EEG measurement function through touch input, the EEG through the sensing means for EEG measurement of the breathing meditation inducing device combined with headphones is measured and then the results is transmitted to the personal terminal or a manager's computer (server),
wherein the EEG measurement and display program is loaded as an application on the personal terminal, and according to the user's selection, it measures EEG or displays the measured result data, and at the same time provides content according to the user's requirements,
wherein when the application is used, the user terminal controls EEG measurement preparation processes so as to be performed the EEG measurement and display functions being provided for a predetermined time, and controls the EEG measurement result data to display the EEG measurement result data in a three-dimensional graph or bar graph form, and when the EEG measurement result is displayed, the comparison data of the past data and the present data is controlled to be displayed on one screen according to the user's request.

4. A breathing mediation induction system for displaying and storing EEG signal according to claim 3,
wherein a user terminal is connected to a personal terminal with a built-in breathing meditation induction device for brain wave measurement and a program that can display and store EEG signals via Bluetooth, and the personal terminal is connected to a computer server for storing EEG signals, and
wherein by operating the app of the personal terminal, the EEG signal is measured, the degree of change is displayed, in the measurement of one's own state through the brain index measurement graph, after a predetermined time after using the psychotherapy device for the brain index measurement bar graph, and the display result can be stored in the computer (server).

5. A breathing mediation induction system for displaying and storing EEG signal according to claim 3,
wherein a user terminal is connected to a personal terminal with a built-in psychotherapy device capable of EEG measurement and a program capable of displaying and storing EEG signals through Bluetooth, and the personal terminal is connected to the EEG signal storage computer server, and
wherein by operating the application of the personal terminal, measuring EEG signals, and grasping one's own state through the brain index measurement bar graph, the brain index measurement bar graph displays the user's mental state into various items by type and when selecting the corresponding item by touch or voice, one or more contents are downloaded thorough a computer (server), and then the specific content can be played by selecting it by touch or voice.

6. A personal terminal for displaying and storing EEG signals in which it includes a Bluetooth, and an application for breathing meditation induction, the application can be downloads through a wireless communication network, and is connected through a wireless communication network and can controls a headphone through a voice or touch input method of the application,
wherein based on the data inputted and transmitted according to the input requirements of the device application, a self-diagnosis process is performed, the result is transmitted as a score through a personal terminal, and content for breathing meditation induction or psychotherapy according to the self-diagnosis score result is provided,
wherein the content is extracted from the database and supplied to the headphone and breathing meditation induction device connected to the personal terminal in a streaming manner,
wherein, in the application through the personal terminal, when the user selects the EEG function through a touch input, the EEG is measured through the EEG sensor of the psychotherapy device and the result is stored in the storage unit of the personal terminal,
wherein the EEG measurement and display program is loaded as an application on the personal terminal, and according to the user's selection, it measures EEG or displays the measured result data, and at the same time provides content according to the user's requirements,
wherein when running the application, and the user executes the EEG measurement preparation process to perform the EEG measurement and display function provided by the app, then controls the EEG measurement to be performed for a predetermined time, and displays the EEG measurement result data as a three-dimensional graph or EEG signal display, when the EEG measurement result is displayed, the comparison data of the past data and the present data is controlled to be displayed on one screen according to the user's request.

7. A personal terminal for displaying and storing EEG signals according to claim 6,
wherein, in the state that the user is connected via Bluetooth to a personal terminal with a built-in breathing meditation induction device and a program that can display and store EEG signals,
wherein the user terminal starts the application of the personal terminal, measures the EEG signal, and determines one's own state through the brain index measurement bar graph, but after using the headset and breathing meditation inducing device for a specific item as a result of the brain index measurement, the brain index after a predetermined time, wherein it is possible to display the degree of change through the measurement graph.

8. A personal terminal for displaying and storing EEG signals according to claim 6,
wherein the items displayed on the brain index measurement bar graph are frequency data measured by the EEG sensing means, and include delta waves, theta waves, alpha waves, SMR waves, M_beta waves, H_beta waves, beta waves, and gamma waves, correspondingly, the brain index bar graph is divided into right brain frequency band and left brain frequency band, respectively, and converted and displayed.

9. A personal terminal for displaying and storing EEG signals according to claim 6,
wherein the items displayed on the brain index measurement bar graph are frequency data measured by the EEG sensing means, and include delta waves, theta waves, alpha waves, SMR waves, M_beta waves, H_beta waves, beta waves, and gamma waves, correspondingly, the brain index bar graph is divided into right brain frequency band and left brain frequency band, respectively, and converted and displayed, and the index graph is displayed as an additional item.

10-17. (canceled)
